(19) United States
(12) Patent Application Publication
(10) Pub. No.: US 2023/0364605 A1
(43) Pub. Date: Nov. 16, 2023

(54) METHODS OF ENGULFING PARTICLES AND DEVICES FOR PRACTICING SAME

(71) Applicant: The Board of Trustees of the Leland Stanford Junior University, Stanford, CA (US)

(72) Inventors: Lambertus Hesselink, Atherton, CA (US); Punnag Padhy, Redwood City, CA (US); Mohammad Zaman, Redwood City, CA (US); Ronald Davis, Palo Alto, CA (US); Michael Jensen, Los Gatos, CA (US)

(21) Appl. No.: 18/141,179

(22) Filed: Apr. 28, 2023

Related U.S. Application Data
(60) Provisional application No. 63/336,963, filed on Apr. 29, 2022.

(51) Int. Cl. B01L 3/00

(52) U.S. Cl. CPC B01L 3/502707; B01L 3/502715; B01L 3/50273; B01L 3/502792; B01L 2200/06; B01L 2300/0654; B01L 2300/0816; B01L 2300/0858; B01L 2300/0877; B01L 2300/12; B01L 2300/165; B01L 2400/0427

(57) ABSTRACT

Provided are methods of engulfing particles into droplets. The methods use a microfluidic device comprising a droplet generator and a chamber, the chamber comprising a liquid medium disposed on a voltage supply electrode and a ground electrode. The methods comprise dispensing a particle and a droplet into the liquid medium, and dielectrophoretically trapping the particle and the droplet using the voltage supply electrode and the ground electrode. The methods further comprise engulfing the particle into the droplet, wherein the engulfing comprises increasing a supply voltage between the voltage supply electrode and the ground electrode, thereby moving the droplet toward the voltage supply electrode and engulfing the particle into the droplet. In certain embodiments, the methods further comprise modifying the engulfed particle, assessing the engulfed particle, or both. Devices that find use in practicing the methods of the present disclosure are also provided.

### CROSS-REFERENCE TO RELATED APPLICATIONS

This application claims the benefit of U.S. Provisional Patent Application No. 63/336,963, filed Apr. 29, 2022, which application is incorporated herein by reference in its entirety.

### STATEMENT OF GOVERNMENT SUPPORT

This invention was made with Government support under contracts HG009758 and GM138716 awarded by the National Institutes of Health. The Government has certain rights in the invention.

### SUMMARY

Provided are methods of engulfing particles into droplets. The methods use a microfluidic device comprising a droplet generator and a chamber, the chamber comprising a liquid medium disposed on a voltage supply electrode and a ground electrode. The methods comprise dispensing a particle and a droplet into the liquid medium, and dielectrophoretically trapping the particle and the droplet using the voltage supply electrode and the ground electrode. The methods further comprise engulfing the particle into the droplet, wherein the engulfing comprises increasing a supply voltage between the voltage supply electrode and the ground electrode, thereby moving the droplet toward the voltage supply electrode and engulfing the particle into the droplet. In certain embodiments, the methods further comprise modifying the engulfed particle, assessing the engulfed particle, or both. Devices that find use in practicing the methods of the present disclosure are also provided.

### BRIEF DESCRIPTION OF THE FIGURES

FIG. 1A-1D: Representation of device and chemical reaction. (A) The device uses dielectrophoretic force to encapsulate and eject a single bead (solid support) from a picoliter reagent droplet. The droplet encapsulates the bead when the supply voltage on the electrode is increased to Vₛ=120 V (amplitude) and ejects the bead when the supply voltage is reduced to Vₛ=0.1 V (amplitude) at 200 Hz supply frequency. Inside the droplet, the fluorescently labelled nucleotides (dCTP-AF647) couple onto the initiator strands on the bead. Complete encapsulation of the solid support provides maximum reagent access to its surface for chemical coupling. Faster reagent diffusion across the microdroplet compared to the bulk scale benchtop platform also improves reagent access to the bead surface. (B) The device footprint depicts the alignment of the droplet generator and the electrodes in the reaction chamber and the spatial location of the bead and the droplet. The dashed black box represents the section of the device represented in part (A). (C) Fabricated silicon-on-glass microfluidic device with suitably aligned ITO electrodes. Purple color is overlayed on the transparent ITO electrodes to distinguish them from the device background. (D) Representation of the chemical reactions on the solid support: (i) Binding the initiator strand on the bead through streptavidin-biotin hydrogen bonds, (ii) Enzymatic coupling of fluorescently labelled nucleotides (dCTP-AF647) onto the 3′ end of the initiator strands on the bead inside the reagent droplet. The bottom box includes the legends of the various components involved in the chemical reaction.

FIG. 2A-2D: Experimental demonstration and numerical simulation of the encapsulation and ejection of a microbead from a picoliter droplet. (A) Experimental implementation and (B) numerical phase field simulation depicts the encapsulation of the bead by the droplet under high supply voltage (Vₛ=120 V) and its ejection from the droplet under low supply voltage (Vₛ=0.1 V). The phase field variable (ϕ) has a value of −1 in the silicone oil suspension medium (phase 1) and a value of 1 in the reagent droplet (phase 2). ϕ transitions from −1 to 1 at the droplet medium interface. The bead is represented in white. (C) Electrocapillary potential energy representation of the engulfing and (D) ejection process. The droplet moves to minimize the total potential energy (U=U_{E}+U_{IT}) of the system. At high Vₛ, the change in electric energy stored between the electrodes is much larger than the change in Gibbs free energy due to the interfacial tension between the bead, droplet and the suspension medium (|ΔU_{E}|>>|ΔU_{IT}|). Therefore, ΔU≈ΔU_{E} decreases as the droplet approaches the electrode and encapsulates the bead. On the other hand, at low Vₛ, change in electric energy is much less than the change in Gibbs free energy (|ΔU_{E}|<<|ΔU_{IT}|). Therefore, ΔU≈ΔU_{IT} decreases as the droplet moves away from the electrode and ejects the bead.

FIG. 3A-3F: Demonstration of chemical coupling and absence of non-specific binding using control. (A) Enzymatic coupling of fluorescently labelled bases (dCTP-AF647) onto the initiator strands on the streptavidin coated fluorescent green microbead. The base and the enzyme (TdT) are present in the reagent droplet. After successful coupling, the (B) fluorescent green (λₑₘ=486 nm) bead under blue excitation (λₑₓ=441 nm) also (C) fluoresces red (λₑₘ=671 nm) under red excitation (λₑₓ=637 nm). (D) Absence of initiator and enzyme in the control experiment ensures no base coupling takes place in the control experiment. The (E) green bead does not fluoresce (F) red. This establishes the absence of any non-specific binding such as physical sticking of the fluorophores on the beads in (C).

FIG. 4A-4D: Fluorescence comparison of reaction fidelity between benchtop platform with a large number of beads packed together and Single Bead-Droplet Solid-Phase Synthesis (SBDSPS). (A) Representative beads (all beads in FIG. 27) show large variation in fluorescence intensity which implies significant variation in the degree of enzymatic coupling between beads. (B) Distribution of average fluorescence intensity of beads reacted on the benchtop shows the relatively small probability of the high fluorescence intensity tail (mean=629.27 and std dev=373). This suggests the correspondingly small probability of finding beads with large coupling efficiencies on benchtop reactors. The fluorescence distribution and the associated statistics were obtained by imaging over 282 representative beads. (C) Three beads reacted inside droplets using SBDSPS yielded high fluorescence intensities corresponding to the low probability tail of the fluorescence intensity distribution of the benchtop reactions in (B). This suggests the absence of bead-bead stacking and faster reagent diffusion in the microdroplet improves reagent access to the bead surface and significantly reduces the probability of obtaining beads with low coupling efficiencies. (D) Comparison of fluorescence intensity between conventionally reacted beads and beads reacted inside droplets using SBDSPS. The bottom graded grey band encompasses the fluorescence intensity distribution of all the 282 representative beads reacted on the benchtop. The bands represent the quartiles of the distribution as well as the fluorescence intensity of beads in the band. The top band represents the fluorescence distribution of the 3 beads reacted using SBDSPS in (C). It spreads over mean−std dev to mean+std dev (mean=2057.5 and std dev=209.05). The dashed black plot in the graded grey band represents the mean of the fluorescence distribution of the beads reacted on the benchtop in (B). The solid black plot in the top grey band represents the average of the fluorescence distribution of the three beads in (C) reacted using SBDSPS. The increased average fluorescence suggests higher reaction fidelity due to improved reagent access to the bead surfaces in the absence of bead-bead stacking. The intensity measurements were taken along the yellow lines in (A) and (C).

FIG. 5: Schematic of the etching process of the silicon part of the device. Layer 1 defines the alignment marks, layer 2 defines the channel, chamber, and ports. Layer 3 etches the chamber and ports deeper, layer 4 etches the fluidic and electrical ports from the backside and layer 5 completes the ports.

FIG. 6: Schematic of the etching process of the glass part of the device. Layer 6 defines the Indium Tin Oxide (ITO) electrodes, layer 7 defines the gold contact pads for external electrical connection.

FIG. 7: Schematic of the alignment and subsequent anodic bonding of silicon and glass parts of the device. The silicon and the glass part of the device are aligned using the alignment markers in an EV Aligner system. After aligning they are bonded anodically using an EV Bonder system.

FIG. 8A-8E: Fabricated silicon and glass components and their subsequent alignment and bonding to obtain the final device. (A) Completely fabricated silicon and (B) glass part. (C) The silicon and the glass part of the device are aligned using the alignment markers in an EV Aligner system. After aligning they are bonded anodically using an EV Bonder system. (D) The top (port) view and the (E) bottom (device) view of the fabricated silicon-on-glass microfluidic device.

FIG. 9A-9F: Optical images depicting the impact of silanization on the contact angle of water droplet on silicon, borofloat glass and Indium Tin Oxide (ITO) surfaces. Oxygen plasma cleaned hydrophilic surface of (A) silicon, (B) glass, and (C) ITO. Silanized hydrophobic surfaces of (D) silicon, (E) glass, and (F) ITO.

FIG. 10: External electrical and fluidic connections made to the device ports on the top side.

FIG. 11A-11B: Fabrication process flowchart. The silicon and glass wafers are processed separately in parallel and then the two wafers are brought together to be anodically bonded. Subsequently they bonded wafers are diced to form device chips, silanized to make the device walls hydrophobic, before sticking the electrical and fluidic connectors to the device ports. Before sticking the connectors, the outer surface of the devices is UV ozone cleaned to strip the silane necessary to glue the fluidic connectors.

FIG. 12A-12B: Device and sample stage. (A) The 3-D printed sample holder is screwed onto the x-y stage which sits on an inverted microscope setup. (B) The device is mounted on the sample holder on top of a glass coverslip to prevent any fluid flow onto the objective.

FIG. 13A-13B: Fluidic subsystem. (A) The piezo-driven pressure controller (OB1 Mk3) from Elveflow drives fluid flow through the microfluidic device. The output of the pressure controller is connected to the oil tank. (B) The pressurized air from the pressure controller drives fluid flow from the oil tank into the device. The device connector connects the output from the oil tank to the device.

FIG. 14A-14B: Electrical subsystem. (A) The function generator generates an electrical signal (up to 15V amplitude) which is then fed to the amplifier which amplifies the signal by 20×. (B) The amplified output of the signal generator is fed to the single pole double throw switch which then connects the supply voltage to either of the electrodes (E₁ or E₂).

FIG. 15A-15B: Optical subsystem. (A) The red light source is used for the fluorescent detection of the labelled nucleotide. (B) The blue light source is used for imaging fluid flow, the fluorescent green beads, and the encapsulation and ejection of the bead by the droplet.

FIG. 16: Interfacial tension measurement between the reagent and the silicone oil suspension medium. Three measurements were taken using the Wilhelmy plate method. Time dependence was captured to account for the contribution of surface adsorption if any on interfacial tension.

FIG. 17A-17B: Addition of 2.5% w/w Span80 to silicone oil enables droplet generation. (A) Droplet generation in the presence of Span80 in the silicone oil suspension medium. Reduced interfacial tension between the reagent and the silicone oil suspension medium as well as the increased contact angle that the reagent forms with the device walls aids in the droplet breakup from the reagent fluid in the microchannel. (B) No droplet generation in the absence of the surfactant as the droplet does not break up from the reagent fluid in the channel.

FIG. 18: Contact angle formed by a reagent phase droplet on a streptavidin coated surface in a medium of 2.5% w/w solution of Span80 in silicone oil. Three time dependent measurements were taken to account for the contribution of surface adsorption if any to contact angle.

FIG. 19A-19B: Electrical and fluidic connections after mounting device on sample stage. (A) Electrical connections made to the three device terminals as shown within the three green circles. (B) Fluidic connection made to the device using the N-333 connectors. The bottom of the tubing is filled with oil to ensure the reagent does not flow into the device as soon as the connection is established for better experimental control. The reagent flows in after the oil. The reagent-oil interface in the input tubing is shown.

FIG. 20: Dielectrophoretic trapping of bead. The bead is trapped dielectrophoretically by applying a voltage of 40 V (amplitude) on the trap electrodes at a supply frequency of 200 Hz. The transparent ITO electrodes appear the same color as the background making it difficult to segregate in the image. Hence, false color has been overlayed (purple) to distinguish the electrode from the rest of the device background.

FIG. 21: Dielectrophoretic trapping of droplet. The droplet is trapped dielectrophoretically by applying a voltage of 15 V (amplitude) on the trap electrodes at a supply frequency of 200 Hz. The transparent ITO electrodes appear the same color as the background making it difficult to segregate in the image. Hence, false color has been overlayed (purple) to distinguish the electrode from the rest of the device background.

FIG. 22A-22B: Focusing the microscope objective on the greater circle of the droplet. (A) Configuration of the bottom glass substrate of the device, electrode, bead, and the droplet. (B) Shifting the focal plane (red dashed line) of the inverted microscope up from the electrode to the greater circle of the droplet. Moving from left to right the bead size increases as the microscope focal plane moves up to focus on the greater circle of the droplet. Experimental data is collected at this focal plane.

FIG. 23A-23F: Fluorescence images for enzymatic coupling experiment on beads with initiator strands and reagent droplet with enzymes. (A) Device filled with oil and with relevant electrical and fluidic connectors before start of experiment with color scale adjusted to lower thresholds and (B) higher thresholds. (A) and (B) are the same image with different color scales. The lower colorscale in (A) is used to highlight the device structure under red illumination while the higher colorscale in (B) is used to depict the absence of any source of noise at the level of the fluorescence signal from the reacted beads due to AF-647 used to label dCTP. (C) Bead before encapsulation and ejection from droplet under blue and (D) red illumination. (E) Bead after encapsulation and ejection from droplet under blue and (F) red illumination. The color of the boxes denotes the illumination source.

FIG. 24: Fluorescence images for enzymatic coupling of dCTP-AF647 to initiator strands on the beads. The images were taken under red illumination (λₑₓ=637 nm) after the beads were ejected out of the reagent droplets.

FIG. 25A-25F: Fluorescence images for control experiment on beads without initiator strands and reagent droplet without enzymes. (A) Device filled with oil and with relevant electrical and fluidic connectors before start of experiment with color scale adjusted to lower thresholds and (B) higher thresholds. (A) and (B) are the same image with different color scales. The lower colorscale in (A) is used to highlight the device structure under red illumination while the higher colorscale in (B) is used to depict the absence of any source of noise at the level of the fluorescence signal from the reacted beads due to AF-647 used to label dCTP. (C) Bead before encapsulation and ejection from droplet under blue and (D) red illumination. (E) Bead after encapsulation and ejection from droplet under blue and (F) red illumination. The color of the boxes denotes the illumination source.

FIG. 26: HPLC chromatogram showing general results for post-synthesis on the benchtop (5 min, 23° C.) free in-solution (T25mer coupled to dCTP-AF647); HPLC analysis was only performed to optimize synthesis conditions before translating to the device. Parameters for sample processing include, Hitachi WAV system, DNA-Sep column (C-18, Cat. DNA-99-3510); method conditions: Buffer A (0.1 M TEAA (triethylammonium acetate) in water), Buffer B (0.1 M TEAA (ADS Biotech), 25% acetonitrile (Sigma Aldrich)), 80° C., 1.2 ml/min, gradient: 82% to 12% A for 10 min; absorbance and fluorescence were measured at 260 nm and 648 nm (excitation)/688 nm (emission), respectfully. Five μl directly aspirated from the reaction were added to the HPLC column.

FIG. 27: Fluorescence images for benchtop implementation of enzymatic coupling reactions for nucleotides. Images were captured as 16-bit Tiff files using PCO.EDGE 5.5 camera with an integration time of 2 s under red excitation (λₑₓ=637 nm) which is indicated by the red outlines around the images. The variation in fluorescence intensities across beads suggests variation in degree of coupling.

FIG. 28A-28B: Fluorescence images for benchtop implementation of the control experiments using beads without initiators and reagents without the enzyme (TDT). Control experiment implemented on beads without initiator strands exposed to coupling reagents without initiator strands shows no red fluorescence under red excitation (λₑₓ=637 nm). The beads fluoresce green under blue excitation. This is used to detect the physical presence of the beads within the field of view of the microscope. The outline color of the images indicates the excitation source (blue λₑₓ—455 nm, red λₑₓ—637 nm).

FIG. 29A-29C: Image processing of benchtop reaction snapshots to extract fluorescence data from beads. (A) A 16-bit snapshot of fluorescent beads post enzymatic coupling of fluorescently labelled nucleotides in benchtop setups under red excitation (λₑₓ=637 nm). It is the same as the first snapshot in FIG. 6A. (B) A binary transformation of the image in (A). (C) Edge and center line detection of the binarized images in (B). The extracted fluorescence intensity patterns across the center lines are plotted in FIG. 4B. The transformations are applied to all the collected bead snapshots.

FIG. 30: Axis symmetric simulation structure. Simulation structure used for the electrohydrodynamic simulation in COMSOL Multiphysics®.

FIG. 31A-31B: Simplistic representation of the evolution of system surfaces and dimensions during bead encapsulation and ejection from the droplet. (A) The surface of the bead is either covered with the droplet or the surrounding medium. (B) Change in the dimension of the droplet with the encapsulation and ejection of the bead from the droplet.

FIG. 32A-32F: Experimental estimation of the contact angle of the reagent droplet on the device surface and walls. (A) Objective focused at the bottom of the droplet on the surface of the device. (B) Diameter of the bottom circle of the droplet. (C) Objective focused at the greater circle of the droplet. (D) Diameter of the greater circle of the droplet. (E) Dimensions associated with the droplet on the device surface. (F) Elementary trigonometric calculation of the contact angle of the droplet on the device surface.

### DETAILED DESCRIPTION

Before the methods and devices of the present disclosure are described in greater detail, it is to be understood that the methods and devices are not limited to particular embodiments described, as such may, of course, vary. It is also to be understood that the terminology used herein is for the purpose of describing particular embodiments only, and is not intended to be limiting, since the scope of the methods and devices will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods and devices. The upper and lower limits of these smaller ranges may independently be included in the smaller ranges and are also encompassed within the methods and devices, subject to any specifically excluded limit in the stated range. Where the stated range includes one or both of the limits, ranges excluding either or both of those included limits are also included in the methods and devices.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods and devices belong. Although any methods and devices similar or equivalent to those described herein can also be used in the practice or testing of the methods and devices, representative illustrative methods and devices are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods and devices are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods and devices, which are, for clarity, described in the context of separate embodiments, may also be provided in combination in a single embodiment. Conversely, various features of the methods and devices, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods and devices and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Methods of Engulfing Particles

Aspects of the present disclosure include methods of engulfing particles into droplets. The methods are implemented using a microfluidic device comprising a droplet generator and a chamber, the chamber comprising a liquid medium disposed on a voltage supply electrode and a ground electrode. The methods comprise dispensing a particle and a droplet into the liquid medium, and dielectrophoretically trapping the particle and the droplet using the voltage supply electrode and the ground electrode. The methods further comprise engulfing the particle into the droplet, wherein the engulfing comprises increasing a supply voltage between the voltage supply electrode and the ground electrode, thereby moving the droplet toward the voltage supply electrode and engulfing the particle into the droplet. The methods find use in a variety of contexts. For example, in some embodiments, the methods further comprise modifying the particle engulfed in the droplet (e.g., conducting solid-phase synthesis on the particle, or the like) where it is demonstrated herein that the present methods provide for significantly higher fidelity modifications as compared to existing approaches and devices (e.g., benchtop platforms) for performing such modifications. Details regarding the methods of the present disclosure will now be described.

The methods of the present disclosure are implemented using a microfluidic device. Non-limiting examples of microfluidic devices that find use in practicing the methods include those described in further detail below in the Devices and Experimental sections of the present disclosure. As used herein, a “microfluidic device” is a device comprising one or more channels comprising dimensions from tens to hundreds of microns. The one or more channels are configured to flow (and in some instances, manipulate and otherwise control) fluids in the range of microliters to picoliters.

The microfluidic device may be made of any suitable material or combination of materials. In certain embodiments, the microfluidic device comprises one or more layers made of a material independently selected from silicon, glass, plastics, and ceramics, including glass ceramics. Suitable plastic materials include, but are not limited to, polycarbonates, polyacrylates such as polymethylmethacrylate (PMMA), and polyethyelenes such as polyethylene terephthalate (PET). Non-limiting examples of suitable glass materials include, but are not limited to, soda-lime silicate, aluminosilicate, alkali-aluminosilicate, borosilicate, alkaliborosilicate, aluminoborosilicate, alkali-aluminoborosilicate, and the like. In some embodiments, the microfluidic device is made of silicon, glass (e.g., borosilicate glass), or a combination thereof. For example, the microfluidic device may be made of a silicon portion bonded to a glass portion. In certain embodiments, the microfluidic device is made of a silicon portion etched to include a droplet generator channel, a reaction chamber and external electrical and fluidic connections, bonded to a glass portion etched to include electrodes and electrical contact points. A non-limiting example of such a device is described in detail in the Experimental section below. According to some embodiments, the height of the chamber is from 100 to 300 μm, e.g., from 125 to 275 μm, from 150 to 250 μm, or from 175 to 225 μm, e.g., about 200 μm. The chamber can have any suitable shape and/or size, such as a rectangle, square, or any other suitable shape, including regular and irregular shapes and shapes with one or more curvilinear edges. In some embodiments, the volume of the chamber is from 2 to 20 mm³, e.g., from 2 to 16 mm³, from 2 to 12 mm³, or from 4 to 8 mm³.

The chamber comprises a liquid medium. In certain embodiments, the liquid medium is inert, comprises a high dielectric strength, or both. By “inert” is meant a liquid that is not generally reactive. As used herein, a liquid having a “high dielectric strength” is a liquid having a dielectric strength of 1×10⁵ V/m or greater. In certain embodiments, the liquid has a dielectric strength of 1×10⁶ V/m or greater, such as 1×10⁷ V/m. According to some embodiments, the liquid medium which is inert and/or has a high dielectric point comprises, consists essentially of, or consists of an oil. Suitable oils include, but are not limited to, silicone oil. In some embodiments, the liquid medium comprises an oil and a surfactant, e.g., a non-ionic surfactant. The surfactant may be present at a suitable percentage (e.g., to reduce the interfacial tension between water and oil), including from 0.5 to 10%, such as from 1 to 5%, e.g., about 2.5%.

The chamber comprises a liquid medium disposed on a voltage supply electrode and a ground electrode. In certain embodiments, the voltage supply electrode is transparent. By “transparent” is meant that the electrode has a transmission of greater than about 80% in the visible region of the spectrum (˜400 to 700 nm). For instance, an exemplary voltage supply electrode may have greater than about 85% transmittance in the visible light range, such as greater than about 90%, or greater than about 95%, including all ranges and subranges therebetween. A non-limiting example of a transparent voltage supply electrode is an indium tin oxide (ITO) voltage supply electrode.

The methods comprise dispensing a particle into the liquid medium of the chamber of the microfluidic device. A “particle” as used herein means a small, localized object to which can be ascribed a physical property such as volume, mass or average size. Particles may accordingly be of a symmetrical, globular, essentially globular or spherical shape, or be of an irregular, asymmetric shape or form. In certain embodiments, the particle is spherical, spheroid, rod-shaped, disk-shaped, pyramid-shaped, cube-shaped, cylinder-shaped, nanohelical-shaped, nanospring-shaped, nanoring-shaped, arrow-shaped, teardrop-shaped, tetrapod-shaped, prism-shaped, or any other suitable geometric or non-geometric shape. The size of a particle may vary. The term “microparticle” refers to particles with a greatest dimension (e.g., diameter) in the nanometer and micrometer range. According to some embodiments, the particle is a bead, e.g., a microbead. As used herein, the term “bead” refers to a small mass that is generally spherical or spheroid in shape. According to some embodiments, a microbead as used herein has an average diameter of from about 0.1 μm to about 500 μm, from about 0.50 μm to about 250 μm, e.g., from about 1 μm to about 100 μm.

Particles (e.g., microparticles, such as microbeads) as defined herein may comprise, consist essentially of, or consist of any suitable material, e.g., they may comprise, consist essentially of, or consist of inorganic or organic material. In certain embodiments, the particle comprises, consists essentially of, or consists of metal or an alloy of metals, or an organic material. According to some embodiments, the particle comprises, consists essentially of, or consists of agarose, polystyrene, latex, polyvinyl alcohol, silica, or a ferromagnetic material (e.g., ferromagnetic metal) metal, alloys or composition materials.

In certain embodiments, the particle is a cell. The cell may be a prokaryotic or eukaryotic cell. According to some embodiments, the cell is an immune cell (e.g., a B cell, a T cell, or the like), an endothelial cell, an epithelial cell, a stem cell, or a neuron.

Any suitable approach may be employed for dispensing a particle into the liquid medium of the chamber of the microfluidic device. In some embodiments, a particle (e.g., microbead) suspended in a solution (e.g., an oil solution) is introduced into the chamber through an inlet. The particle concentration may be tuned to ensure a single particle enters the vicinity of the electrodes and is trapped dielectrophoretically.

Similarly, any suitable approach may be employed for dispensing the droplet into the liquid medium of the chamber of the microfluidic device. In some embodiments, the microfluidic device comprises a droplet generator channel adapted to dispense a single droplet into the chamber at a time. According to some embodiments, the droplet generator channel is from 10 to 40 μm wide, such as from 15 to 35 μm wide, or from 20 to 30 μm wide, e.g., about 25 μm wide. The droplet generator channel may be operably coupled to a fluidic port which is in turn operably coupled to a fluidic subsystem comprising a pressure controller (e.g., a piezo driven pressure controller) that allows for the dispensing of single droplets via the droplet generator channel into the chamber. In certain embodiments, a pressure pulse of from 1 to 20 mbar (e.g., from 5 to 15 mbar, e.g., about 10 mbar) is exerted using the pressure controller to dispense a single droplet into the chamber. In some embodiments, the volume of the droplet generated by the droplet generator (e.g., droplet generator channel) is from 10 to 100 picoliters, e.g., from 20 to 80 picoliters, such as from 40 to 60 picoliters.

As summarized above, the methods of the present disclosure comprise dielectrophoretically trapping the particle and the droplet. In certain embodiments, the particle is dispensed first into the chamber, and once a particle is on or near the voltage supply electrode, the voltage supply is switched on to dielectrophoretically trap the bead. For example, the voltage supply may be switched on and set to a suitable amplitude and frequency to dielectrophoretically trap the particle. After trapping the particle, the voltage supply may be switched off, followed by the dispensing of a single droplet into the chamber. The electrodes in the chamber are suitably aligned with the droplet generator channel (e.g., the voltage supply electrode may be from 50 to 150 μm from the opening of the droplet generator channel, such as from 75 to 125 μm from the opening of the droplet generator channel) to ensure that the generated droplet lies within their dielectrophoretic trapping range. Next, the voltage supply may be switched on again and a suitable voltage is applied to trap the droplet adjacent to the particle on or near the voltage supply electrode. A suitable voltage for trapping the droplet adjacent to the particle on or near the voltage supply electrode is one that produces an electric field strength of from 1e+3 to 1e+4 V/m, for example.

The methods further comprise engulfing the particle into the droplet, where the engulfing comprises increasing a supply voltage between the voltage supply electrode and the ground electrode, thereby moving the droplet toward the voltage supply electrode and engulfing the particle into the droplet. According to some embodiments, and continuing on from the preceding paragraph, the supply voltage may be increased, causing the droplet to move toward the voltage supply electrode to engulf the particle. A suitable increased voltage for engulfing the particle into the droplet is one that produces an electric field strength of from 1e+5 to 1e+7 V/m (e.g., about 1e+6), for example.

In certain embodiments, the methods further comprise ejecting the engulfed particle from the droplet, e.g., by reducing the supply voltage. A suitable reduced voltage for ejecting the particle from the droplet is one that produces an electric field strength of from 1e+3 to 1e+4 V/m, for example.

According to some embodiments, once the particle is engulfed into the droplet, the methods further comprise modifying the engulfed particle. For example, the droplet may be a reagent droplet comprising one or more reagents for effecting a desired modification of the particle. In one non-limiting example, the modification comprises solid phase synthesis, where one or more steps of a solid phase synthesis (e.g., enzymatic or chemical addition of a nucleotide or polynucleotide, or enzymatic or chemical addition of an amino acid or polypeptide) are performed on the particle, e.g., a microparticle, such as a microbead or the like.

In certain embodiments, when the method further comprises modifying the engulfed particle, the modifying comprises covalently or non-covalently attaching a molecule present in the droplet to the particle. In some embodiments, the surface of the particle may be functionalized (or “activated”/“derivatized”) with reactive groups to which the molecule present in the droplet is attached. For example, the surface may be functionalized with any useful/convenient reactive group, including but not limited to thiol groups (—SH), amine groups (—NH2), carboxyl groups (—COO), carbodiimide crosslinker chemistry, polyethylene glycol (PEG), and/or the like. Bioconjugation strategies that find use in attaching a molecule present in the droplet to a reactive group on the particle are described in Hermanson, “Bioconjugate Techniques,” Academic Press, 2nd edition, Apr. 1, 2008, Haugland, 1995, Methods Mol. Biol. 45:205-21; Brinkley, 1992, Bioconjugate Chemistry 3:2, and elsewhere.

According to embodiments in which the particle comprises silica (SiO₂) on its surface, the surface silanol groups may be functionalized. The silica can be functionalized to bear free thiol/sulfhydryl groups either during the course of making the SiO₂ surface (“direct modification”), or after the particle has been completely formed (“post-modification”). Reactions for the modification of silanol groups are known and include, but are not limited to, modification of the SiO₂ surface to present amines (e.g., by reaction with aminopropyl trimethoxysilane (APTMS)) or ethoxides (e.g., by reaction with 3-glycidyloxypropyl-trimethoxysilane (GPTMS)). Reagents also exist to incorporate sulfhydryls, carboxyl and other useful reactive groups for conjugation.

The molecule present in the droplet may already include a functional group useful for reacting with a reactive group present on the particle, or such a functional group may be provided to the molecule. Functional groups that may be used to bind a molecule present in the droplet to the particle include, but are not limited to, active esters, isocyanates, imidoesters, hydrazides, amino groups, aldehydes, ketones, photoreactive groups, maleimide groups, alpha-halo-acetyl groups, epoxides, azirdines, and the like. Reagents such as iodoacetamides, maleimides, benzylic halides and bromomethylketones react by S-alkylation of thiols to generate stable thioether products. For example, at pH 6.5-7.5, maleimide groups react with sulfhydryl groups to form stable thioether bonds. Arylating reagents such as NBD halides react with thiols or amines by a similar substitution of the aromatic halide by the nucleophile. Because the thiolate anion is a better nucleophile than the neutral thiol, cysteine is more reactive above its pKₐ (˜8.3, depending on protein structural context). Thiols also react with certain amine-reactive reagents, including isothiocyanates and succinimidyl esters. The TS-Link series of reagents are available for reversible thiol modification.

With respect to amine reactive groups, primary amines exist at the N-terminus of polypeptide chains and in the side-chain of lysine (Lys, K) amino acid residues. Among the available functional groups in typical biological or protein samples, primary amines are especially nucleophilic, making them ready targets for conjugation with several reactive groups. For example, NHS esters are reactive groups formed by carbodiimide-activation of carboxylate molecules. NHS ester-activated crosslinkers and labeling compounds react with primary amines in physiologic to slightly alkaline conditions (pH 7.2 to 9) to yield stable amide bonds. The reaction releases N-hydroxysuccinimide (NHS). Also by way of example, imidoester crosslinkers react with primary amines to form amidine bonds. Imidoester crosslinkers react rapidly with amines at alkaline pH but have short half-lives. As the pH becomes more alkaline, the half-life and reactivity with amines increases. As such, crosslinking is more efficient when performed at pH 10 than at pH 8. Reaction conditions below pH 10 may result in side reactions, although amidine formation is favored between pH 8-10.

Numerous other synthetic chemical groups will form chemical bonds with primary amines, including but not limited to, isothiocyanates, isocyanates, acyl azides, sulfonyl chlorides, aldehydes, glyoxals, epoxides, oxiranes, carbonates, aryl halides, carbodiimides, anhydrides, and fluorophenyl esters. Such groups conjugate to amines by either acylation or alkylation.

According to some embodiments, the modifying comprises covalently attaching a nucleotide or polynucleotide present in the droplet to the particle. The term “nucleotide” is intended to include those moieties that contain not only the naturally occurring purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like. As used herein, an “oligonucleotide” is a single-stranded multimer of nucleotides from 2 to 500 nucleotides, e.g., 2 to 200 nucleotides. Oligonucleotides may be synthetic or may be made enzymatically, and, in some embodiments, are 5 to 50 nucleotides in length (e.g., 9 to 50 nucleotides in length). Oligonucleotides may contain ribonucleotide monomers (i.e., may be oligoribonucleotides or “RNA oligonucleotides”) or deoxyribonucleotide monomers (i.e., may be oligodeoxyribonucleotides or “DNA oligonucleotides”). Oligonucleotides may be 5 to 9, 10 to 20, 21 to 30, 31 to 40, 41 to 50, 51 to 60, 61 to 70, 71 to 80, 80 to 100, 100 to 150 or 150 to 200, up to 500 or more nucleotides in length, for example. The term “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, greater than 10,000 bases, greater than 100,000 bases, greater than about 1,000,000, up to about 10¹⁰ or more bases composed of nucleotides, e.g., deoxyribonucleotides or ribonucleotides, which can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine, thymine, uracil (G, C, A, T and U respectively). DNA and RNA have a deoxyribose and ribose sugar backbone, respectively, whereas PNA's backbone is composed of repeating N-(2-aminoethyl)-glycine units linked by peptide bonds. In PNA various purine and pyrimidine bases are linked to the backbone by methylenecarbonyl bonds. A locked nucleic acid (LNA), often referred to as inaccessible RNA, is a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired. The term “unstructured nucleic acid,” or “UNA,” is a nucleic acid containing non-natural nucleotides that bind to each other with reduced stability. For example, an unstructured nucleic acid may contain a G′ residue and a C′ residue, where these residues correspond to non-naturally occurring forms, i.e., analogs, of G and C that base pair with each other with reduced stability, but retain an ability to base pair with naturally occurring C and G residues, respectively. Unstructured nucleic acid is described in US20050233340, which is incorporated by reference herein for disclosure of UNA.

When the modifying comprises covalently attaching a nucleotide or polynucleotide present in the droplet to the particle, the nucleotide or polynucleotide may be attached to the particle chemically. Approaches for chemical attachment of nucleotides or polynucleotides to the particle include, but are not limited to, click-chemistry based approaches. Click-chemistry reactions that may be employed include (i) nucleophilic substitutions; (ii) additions to C—C multiple bonds (e.g., Michael addition, epoxidation, dihydroxylation, aziridination); (iii) nonaldol like chemistry (e.g., N-hydroxysuccinimide active ester couplings); and (iv) cycloadditions (e.g., Diels-Adler reaction, Huisgen's cycloaddition). Huisgen's cycloaddition has been applied in various branches of chemistry. It consists of the condensation of organic azides with alkyne groups to form 1,2,3-triazole linkages. Azide and alkyne functionalities can be easily introduced in the scaffold of large organic constructs of biological relevance. The reaction may be catalyzed by introducing copper(I). The Cu(I) core has a dual effect in that it activates the slow-reacting alkyne group thus accelerating the azide-alkyne condensation kinetics by ˜10⁷-10⁸-fold, and it organizes the reacting groups by “templation” so that only a regiospecific 1,4-disubstituted adduct is formed. This reaction is known as the copper-catalyzed azide alkyne cycloaddition (CuAAC), and its compatibility with a wide range of biological substrates and synthetic conditions makes CuAAC the flagship among click conjugations. Since its discovery, Cu(I)-catalyzed azide alkyne cycloaddition has been widely used within the fields of biology, biochemistry, and biotechnology. Click-chemistry reactions that may be employed to attach the nucleotide or polynucleotide to the particle include, but are not limited to, Huisgen Azide-Alkyne 1,3-Dipolar Cycloaddition, Copper-Catalyzed Azide-Alkyne Cycloaddition (CuAAC), Ruthenium-Catalyzed Azide-Alkyne Cycloaddition (RuAAC), and the like. Details regarding click-chemistry with nucleic acids are found, e.g., in Fantoni et al. (2021) Chem. Rev. 121(12):7122-7154.

When the modifying comprises covalently attaching a nucleotide or polynucleotide present in the droplet to the particle, the nucleotide or polynucleotide may be attached to the particle enzymatically. Enzymatic attachment may be performed by providing, e.g., a suitable nucleic acid ligase (e.g., a DNA ligase) and conditions suitable for enzymatic ligation reactions. Enzymatic attachment may also be performed by providing, e.g., a suitable nucleic acid polymerase and conditions suitable for polymerase-mediated extension reactions (e.g., extension of a primer disposed on the particle). Extension reactions where a nucleic acid present on the engulfed particle is extended within the droplet may be templated or non-templated. Enzymes that find use in templated extension reactions include, e.g., polymerases. Enzymes that find use in non-templated extension reactions include, e.g., terminal transferases. Terminal transferase (TdT) is a template independent polymerase that catalyzes the addition of deoxynucleotides to the 3′ hydroxyl terminus of DNA molecules.

When the modifying comprises non-covalently attaching a nucleotide or polynucleotide present in the droplet to the particle, in some embodiments, the attaching comprises base-pairing the nucleotide or polynucleotide to a nucleotide or polynucleotide present on the particle. Such base-pairing will typically be performed under hybridization conditions. As used herein, the term “hybridization conditions” means conditions in which a nucleic acid or oligonucleotide specifically hybridizes to a nucleotide or polynucleotide present on the particle. Whether such hybridization occurs is determined by such factors as the degree of complementarity and the temperature at which the hybridization occurs, which may be informed by the melting temperature (T_{M}). The melting temperature refers to the temperature at which half of the nucleic acid duplexes remain hybridized and half of the duplexes dissociate into single strands. The Tm of a duplex may be experimentally determined or predicted using the following formula Tm=81.5+16.6(log 10[Na+])+0.41 (fraction G+C)−(60/N), where N is the chain length and [Na+] is less than 1 M. See Sambrook and Russell (2001; Molecular Cloning: A Laboratory Manual, 3rd ed., Cold Spring Harbor Press, Cold Spring Harbor N.Y., Ch. 10). Other more advanced models that depend on various parameters may also be used to predict Tm of duplexes depending on various hybridization conditions. Approaches for achieving specific nucleic acid hybridization may be found in, e.g., Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier (1993). When the modifying comprises non-covalently attaching a nucleotide or polynucleotide present in the droplet to the particle, in some embodiments, the nucleotide or polynucleotide is attached to the particle via a biotin-streptavidin interaction.

According to some embodiments, the modifying comprises covalently attaching an amino acid or polypeptide present in the droplet to the particle. The term “amino acid” generally refers to any monomer unit that comprises a substituted or unsubstituted amino group, a substituted or unsubstituted carboxy group, and one or more side chains or groups, or analogs of any of these groups. Exemplary side chains include, e.g., thiol, seleno, sulfonyl, alkyl, aryl, acyl, keto, azido, hydroxyl, hydrazine, cyano, halo, hydrazide, alkenyl, alkynl, ether, borate, boronate, phospho, phosphono, phosphine, heterocyclic, enone, imine, aldehyde, ester, thioacid, hydroxylamine, or any combination of these groups. Other representative amino acids include, but are not limited to, amino acids comprising photoactivatable cross-linkers, metal binding amino acids, spin-labeled amino acids, fluorescent amino acids, metal-containing amino acids, amino acids with novel functional groups, amino acids that covalently or noncovalently interact with other molecules, photocaged and/or photoisomerizable amino acids, radioactive amino acids, amino acids comprising biotin or a biotin analog, glycosylated amino acids, other carbohydrate modified amino acids, amino acids comprising polyethylene glycol or polyether, heavy atom substituted amino acids, chemically cleavable and/or photocleavable amino acids, carbon-linked sugar-containing amino acids, redox-active amino acids, amino thioacid containing amino acids, and amino acids comprising one or more toxic moieties. As used herein, the term “amino acid” includes the following twenty natural or genetically encoded alpha-amino acids: alanine (Ala or A), arginine (Arg or R), asparagine (Asn or N), aspartic acid (Asp or D), cysteine (Cys or C), glutamine (Gin or Q), glutamic acid (Glu or E), glycine (Gly or G), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

In certain embodiments, the amino acid or polypeptide is attached to the particle chemically or enzymatically. When the amino acid or polypeptide is attached to the particle chemically, in certain embodiments, the attachment utilizes any of the click-chemistry approaches described above, e.g., Huisgen Azide-Alkyne 1,3-Dipolar Cycloaddition, Copper-Catalyzed Azide-Alkyne Cycloaddition (CuAAC), Ruthenium-Catalyzed Azide-Alkyne Cycloaddition (RuAAC), and the like. Details regarding click-chemistry with amino acids and polypeptides are found, e.g., in Parker & Pratt (2020) Cell 180(4):605-632. When the amino acid or polypeptide is attached to the particle chemically, in certain embodiments, the attachment utilizes solid phase peptide chemistry, e.g., standard 9-fluorenylmethyloxycarbonyl (Fmoc)- or Boc-based solid phase peptide chemistry may be employed.

According to some embodiments, the modifying comprises non-covalently attaching an amino acid or polypeptide present in the droplet to the particle. In one non-limiting example, the amino acid or polypeptide is attached to the particle via a biotin-streptavidin interaction. Also by way of example, non-covalently attaching an amino acid or polypeptide present in the droplet to the particle comprised binding an antibody to the particle (e.g., a cell, a microbead, or the like).

According to some embodiments, the modifying comprises covalently or non-covalently attaching a sugar or carbohydrate present in the droplet to the particle.

In certain embodiments, the particle comprises a protein on its surface, and the modifying comprises modifying the protein. Such protein modifications include, but are not limited to, post-translational modifications (PTMs) that occur in nature such as glycosylation, phosphorylation, ubiquitination, nitrosylation, methylation, acetylation, lipidation, and/or the like. By way of example, the modifying may comprise glycosylating the protein with a carbohydrate present in the droplet. Glycosylation is the enzymatic post-translational addition of carbohydrates (glycans) to proteins and lipids, resulting in “glycoproteins” and “glycolipids,” respectively. Canonically, glycoprotein glycans can be N-linked (linkage to the amide group of Asn) or O-linked (linkage to the hydroxyl group of Ser, Thr). The particular glycan structures, the “glycoforms,” of a glycoprotein impact the function, stability, folding, localization and ligand specificity of the glycoprotein, and play a role in cell adhesion and cell trafficking by modulating how cells interact with each other and with their extracellular matrix environment. Also by way of example, the modifying may comprise phosphorylating the protein with a phosphate group present in the droplet, e.g., the droplet may comprise a kinase enzyme, phosphate groups, an appropriate buffer, etc. to carry out phosphorylation of the protein within the droplet.

According to some embodiments, once the particle is engulfed into the droplet, the methods further comprise assessing the engulfed particle. In some embodiments, the methods comprise assessing the engulfed particle prior to and/or subsequent to modifying the engulfed particle. In some embodiments, the engulfed particle is assessed to determine whether the particle comprises the desired modification.

In certain embodiments, the particle is a cell, and the method comprises assessing the cell for expression of a polypeptide. The polypeptide may be a cytoplasmic polypeptide, a cell surface polypeptide, a secreted polypeptide, or the like. According to some embodiments, the polypeptide is an antibody. In certain embodiments, the droplet comprises a binding partner for the polypeptide, and wherein the assessing comprises assessing for complexes comprising the binding partner and the polypeptide. Non-limiting examples of such binding partners include an antibody, a ligand, an aptamer, an antigen, or a small molecule.

By “antibody” is meant an antibody or immunoglobulin of any isotype (e.g., IgG (e.g., IgG1, IgG2, IgG3, or IgG4), IgE, IgD, IgA, IgM, etc.), whole antibodies (e.g., antibodies composed of a tetramer which in turn is composed of two dimers of a heavy and light chain polypeptide); single chain antibodies (e.g., scFv); fragments of antibodies (e.g., fragments of whole or single chain antibodies) which retain specific binding to the cell surface molecule of the target cell, including, but not limited to single chain Fv (scFv), Fab, (Fab′)₂, (scFv′)₂, and diabodies; chimeric antibodies; monoclonal antibodies, human antibodies, humanized antibodies (e.g., humanized whole antibodies, humanized half antibodies, or humanized antibody fragments, e.g., humanized scFv); and fusion proteins comprising an antigen-binding portion of an antibody and a non-antibody protein. In certain embodiments, the antibody is selected from an IgG, single chain Fv (scFv), Fab, (Fab)₂, (scFv′)₂, or a single variable domain located on a heavy chain (VHH). According to some embodiments, the antibody is a VHH (sometimes referred to herein and elsewhere as a “nanobody”).

In certain embodiments, the particle is a B cell, and the method comprises assessing the B cell for expression of an antibody that specifically binds an antigen present in the droplet.

As used herein, a “ligand” is a substance that forms a complex with a biomolecule in nature to serve a biological purpose. The ligand may be a substance selected from a circulating factor, a secreted factor, a cytokine, a growth factor, a hormone, a peptide, a polypeptide, a small molecule, and a nucleic acid, that forms a complex, e.g., with a cell surface molecule on the surface of the cell. In certain embodiments, the ligand is modified in such a way that complex formation with the cell surface molecule occurs, but the normal biological result of such complex formation does not occur. In certain embodiments, the ligand is the ligand of a cell surface receptor present on the cell. Cell surface receptors of interest include, but are not limited to, receptor tyrosine kinases (RTKs), non-receptor tyrosine kinases (non-RTKs), growth factor receptors, etc.

By “aptamer” is meant a nucleic acid (e.g., an oligonucleotide) that has a specific binding affinity for the cell surface molecule. Aptamers exhibit certain desirable properties, such as ease of selection and synthesis, high binding affinity and specificity, low immunogenicity, and versatile synthetic accessibility. Aptamers that bind to cell surface molecules are known and include, e.g., TTA1 (a tumor targeting aptamer to the extracellular matrix protein tenascin-C).

By “small molecule” is meant a compound having a molecular weight of 1000 atomic mass units (amu) or less. In some embodiments, the small molecule is 750 amu or less, 500 amu or less, 400 amu or less, 300 amu or less, or 200 amu or less. In certain aspects, the small molecule is not made of repeating molecular units such as are present in a polymer. In certain aspects, the cell surface molecule is a receptor for which the ligand is a small molecule, and the small molecule is the small molecule ligand (or a derivative thereof) of the receptor.

As noted above, in certain embodiments, the methods further comprise ejecting the engulfed particle from the droplet, e.g., by reducing the supply voltage. According to some embodiments, the supply voltage is reduced to from 0.01 to 1V amplitude (e.g., from 0.05 to 0.5V amplitude, such as to about 0.1V amplitude) to eject the engulfed particle from the droplet. In certain embodiments, the ejected particle is further subjected to one or more of the dispensing, dielectrophoretically trapping and/or engulfing steps. For example, in some embodiments, an engulfed particle is modified (e.g., any of the modifications described herein), ejected from the droplet, dielectrophoretically trapped with the same or different droplet (e.g., a same or different reagent droplet), and engulfed into the same or different droplet, e.g., by increasing the supply voltage as described herein. In certain embodiments, the particle engulfed into the same or different droplet is further modified within the same or different droplet. As will be appreciated with the benefit of the present disclosure, such iterative modifications may be performed according to the methods and devices of the present disclosure to carry out, e.g., solid-phase synthesis (non-limiting examples of which include chemical and/or biological combinatorial synthesis, and the like), DNA sequencing (e.g., sequencing by synthesis), etc. on a single particle.

Accordingly, and with the benefit of the present disclosure, it will be appreciated that the present methods and devices find use in a variety of applications including but not limited to the following:
1. Enzymatic and/or chemical substrate ligation (e.g., DNA ligase)
   Initiator strand—primer for terminal transferase
   Cu(I)-catalyzed azide-alkyne cycloaddition (CuAAC)
2. Enzyme-linked immunosorbent assay
   antigen on the particle; or antibody on the particle
3. Biological/chemical combinatorial synthesis
4. Combinatorial library generation (e.g., one particle one compound (OBOC) and split pool)
5. High-throughput screening (e.g., drug discovery, substrate toxicity)
   different compounds in different droplets
6. Hybridization assay (e.g., sandwich, competitive, nuclease, dual ligation)
   hybridize nucleic acid in droplet to nucleic acid on particle;
   error detection (quality control for sequence built onto the particle)
      assess for hybridization under different conditions (temperature and/or salt concentration)
      oligonucleotide error filtration (e.g., mismatches, deletions)
7. Oligonucleotide read/write synthesis (e.g., DNA, RNA, peptide)
   fluorescent readout with information regarding which nucleotide was added
8. Antibody capture (e.g., Protein-G, affinity)
   e.g., protein-G on particle to capture IgG
   bind a particular class of antibody onto the particle
9. Particle surface functionalization (e.g., carbodiimide crosslinker chemistry, PEG)
   NHS, etc.
10. Polymerase chain reaction (see also #16)
   primer on particle; template and reverse primer in reagent droplet; or bridge-type amplification
11. Protein modification (e.g., phosphorylation, methylation, glycosylation)
   protein on particle, modification moiety in reagent droplet
12. DNA Sequencing
13. Biological cell sorting and manipulation
   isolate particular cell types (immune cells, red blood cells, etc.)
      ligand or receptor on particle for binding partner on cell
14. Substrate/oligonucleotide enzymatic functionalization (e.g., TdT+3′ azide coupling)
15. Oligonucleotide error filtration (e.g., mismatches, deletions)
16. Polymerase chain assembly (e.g., oligonucleotide tiling and amplification)

Any embodiments of the methods of the present disclosure may comprise imaging the particle during one or more, or each of, the dispensing, dielectrophoretically trapping, and engulfing steps. Imaging may also be performed during any modification and/or assessment steps. For example, imaging may be performed to monitor and/or confirm that a desired modification of the particle has occurred. In some embodiments, assessing the engulfed particle (e.g., performing any of the assessments described herein) comprises imaging the droplet, the particle therein, or both. An example approach for imaging particles and droplets is described in detail in the Experimental section herein. For example, the device may be operably coupled to an optical subsystem comprising a microscope, one or more suitable excitation sources (e.g., LED, laser, and/or the like), optical filters, a camera, and any other useful components for imaging the droplet, the particle therein, or both.

### Microfluidic Devices

Aspects of the present disclosure further include microfluidic devices. The microfluidic devices find use in a variety of contexts, including for practicing the methods of the present disclosure.

In certain embodiments, a microfluidic device of the present disclosure comprises at least one of a chamber adapted to contain a liquid medium; a fluidic port operably coupled to the chamber, a droplet generator operably coupled to the chamber, and a voltage supply electrode and a ground electrode disposed on a surface of the chamber. As used herein, “operably coupled” means that the two elements are in a functional relationship with each other. The voltage supply electrode and a ground electrode are aligned with the droplet generator for controlled manipulation of a droplet and a particle when the device is in use. The electrodes in the chamber are suitably aligned with the droplet generator channel (e.g., the voltage supply electrode may be from 50 to 150 μm from the opening of the droplet generator channel, such as from 75 to 125 μm from the opening of the droplet generator channel) to ensure that a generated droplet lies within a dielectrophoretic trapping range when the device is in use.

The microfluidic device may be made of any suitable material or combination of materials. In certain embodiments, the microfluidic device comprises one or more layers made of a material independently selected from silicon, glass, plastic, and ceramic, including glass ceramic. Suitable plastic materials include, but are not limited to, polycarbonates, polyacrylates such as polymethylmethacrylate (PMMA), and polyethyelenes such as polyethylene terephthalate (PET). Non-limiting examples of suitable glass materials include, but are not limited to, soda-lime silicate, aluminosilicate, alkali-aluminosilicate, borosilicate, alkaliborosilicate, aluminoborosilicate, alkali-aluminoborosilicate, and the like. In some embodiments, the microfluidic device is made of silicon, glass (e.g., borosilicate glass), or a combination thereof. For example, the microfluidic device may be made of a silicon portion bonded to a glass portion. In certain embodiments, the microfluidic device is made of a silicon portion etched to include a droplet generator channel, a reaction chamber and external electrical and fluidic connections, bonded to a glass portion etched to include electrodes and electrical contact points. A non-limiting example of such a device is described in detail in the Experimental section below. According to some embodiments, the height of the chamber is from 100 to 300 μm, e.g., from 125 to 275 μm, from 150 to 250 μm, or from 175 to 225 μm, e.g., about 200 μm. The chamber can have any suitable shape and/or size, such as a rectangle, square, or any other suitable shape, including regular and irregular shapes and shapes with one or more curvilinear edges. In some embodiments, the volume of the chamber is from 2 to 20 mm³, e.g., from 2 to 16 mm³, from 2 to 12 mm³, or from 4 to 8 mm³.

The chamber comprises a voltage supply electrode and a ground electrode disposed on a surface thereof. In certain embodiments, the voltage supply electrode is transparent. A non-limiting example of a transparent voltage supply electrode is an indium tin oxide (ITO) voltage supply electrode.

In some embodiments, the droplet generator comprises a droplet generator channel adapted to dispense a single droplet into the chamber at a time. According to certain embodiments, the droplet generator channel is from 10 to 40 μm wide, such as from 15 to 35 μm wide, or from 20 to 30 μm wide, e.g., about 25 μm wide.

A microfluidic device of the present disclosure may be operably coupled to one or more subsystems. For example, the microfluidic device may be operably coupled to one or more of a fluidic subsystem, electrical subsystem, and/or an optical subsystem. Such subsystems may facilitate use of the device, e.g., for practicing the methods of the present disclosure. Non-limiting examples of such subsystems are described in detail in the Experimental section below. In some embodiments, a fluidic subsystem comprises a fluidic tank comprising an output fluidically coupled to the chamber when the device is in use. A fluidic subsystem may also comprise a pressure controller operably coupled to a fluidic tank. According to some embodiments, the voltage supply electrode and the ground electrode are operably coupled to an electrical subsystem, e.g., an electrical subsystem comprising a function generator and an amplifier. In certain embodiments, the device is operably coupled to an optical subsystem comprising a microscope. An optical subsystem may further comprise one or more suitable excitation sources (e.g., LED, laser, and/or the like), optical filters, a camera, and any other useful components for imaging the droplet, the particle therein, or both.

The following examples are offered by way of illustration and not by way of limitation.

### EXPERIMENTAL

### Dielectrophoretic Bead-Droplet Reactor for High-Fidelity Solid-Phase Enzymatic DNA Synthesis

Synthetic DNA is indispensable for research in synthetic biology with widespread applications spanning healthcare (1, 2), energy and environment (2, 3), agriculture (1, 4), bio- and nano-materials (1, 2, 5), and data storage (6, 7). Hence, there is a continuous increase in demand for accurate, low-cost, high-throughput, and decentralized synthesis of long oligonucleotides (single stranded DNA) to fuel the ever-expanding frontiers of synthetic biology and its applications. However, even after decades of advancement and optimization, state-of-the-art automated solid-phase synthesizers implementing phosphoramidite or enzymatic reaction cycles are limited to oligonucleotides that are 200-300 bases (dNTPs) long (8-12). The intended full-length product (FLP) yield is merely around 33% due to accumulated errors over multiple reactions. Thus, depending on the sensitivity of the application, expensive and time-consuming methods of purification/error correction are often required to isolate the FLP post synthesis, which further reduce the overall yield and escalate the cost of synthesis per base (8-10, 12-13). Moreover, such purification methods become increasingly ineffective with the increase in the oligonucleotide strand length (13). Therefore, it is imperative to maximize the fidelity of individual reactions to limit the accumulated errors for the synthesis of long strands of oligonucleotides with practical yields.

Inadequate exposure of reaction sites to reagents on the synthesis platforms gives rise to error accumulation which ultimately limits the practically attainable strand length, irrespective of the reaction chemistry. In microarrays, misalignment of optical beams or reagent droplets with respect to synthesis spots leads to reaction errors (8-10, 13). Design improvements have resulted in reaction efficiencies comparable to columns (10). In column-based benchtop platforms with many beads pooled together, bead-bead stacking fundamentally reduces the bead surface-to-reagent ratio; thus, restricting the fidelity of reactions (8-10). For example, inadequate exposure to reagents leads to large polydispersity in enzymatically synthesized oligonucleotides (14). While research on oligonucleotide synthesis is primarily focused on reliable, high-throughput, low-cost and massively parallel production of many distinct oligo sequences using either the phosphoramidite or enzymatic approaches (12), the problem of reaction error accumulation due to the synthesis platforms remains largely unsolved. Hence, radically new physical approaches to implement solid-phase synthesis (15) are required to overcome this long-standing challenge plaguing oligonucleotide synthesis and other forms of solid-phase synthesis in general (15).

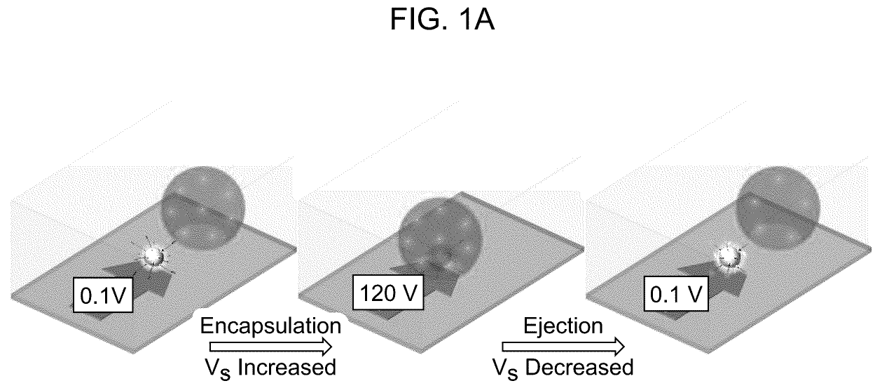

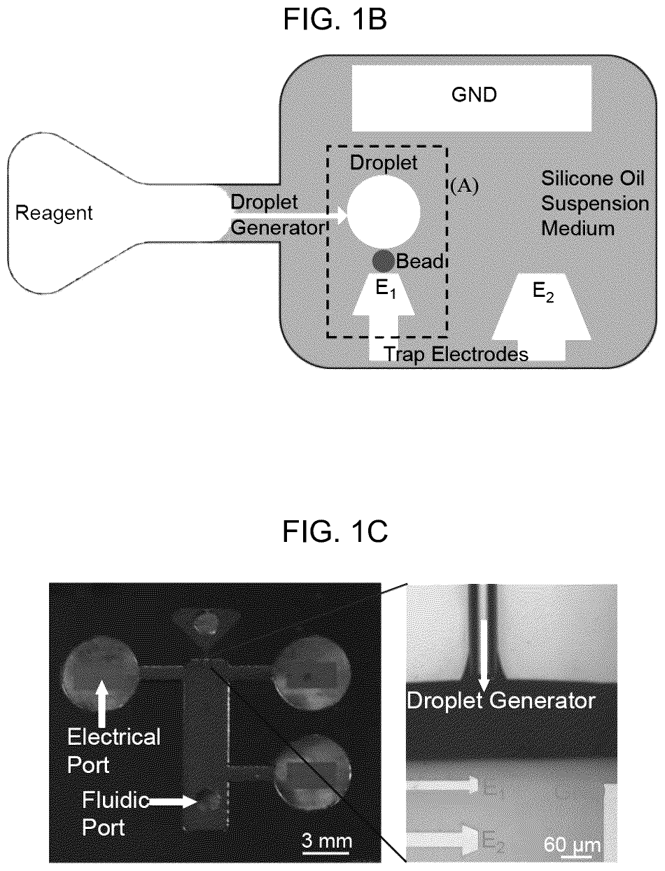

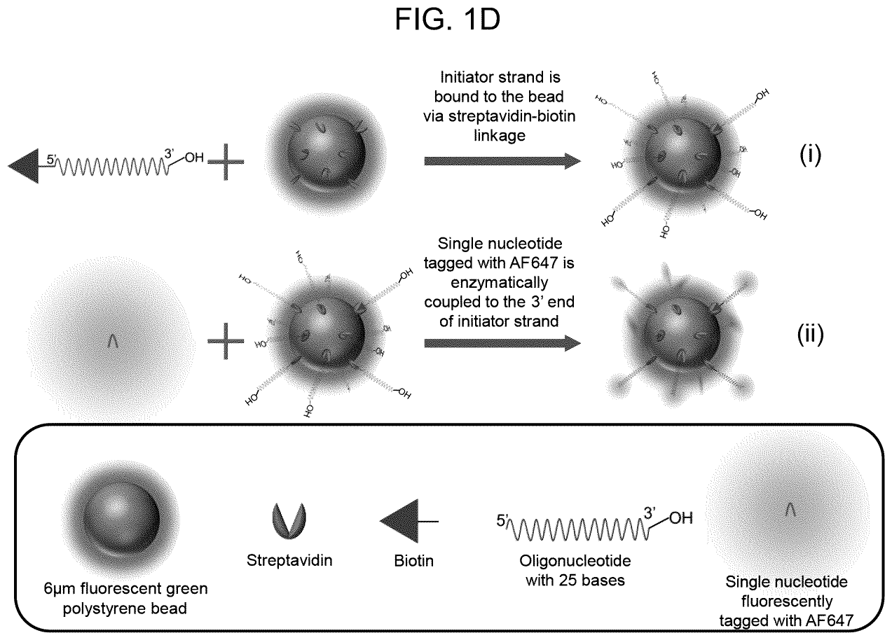

Introduced herein is a method sometimes referred to herein as Single Bead-Droplet Solid-Phase Synthesis (SBDSPS), a novel technique to synthesize on isolated beads to circumvent bead-bead stacking and maximize bead surface-to-reagent ratio and fidelity of reactions. Picoliter reagent droplets are used as microreactors to minimize reagent consumption (16). Reaction is implemented by encapsulating and ejecting the bead from the droplet. Capillary forces due to interfacial tension are significant at this length scale and must be overcome to manipulate the bead across the droplet-medium interface. All prior bead loading techniques employ flow-based approaches to introduce a single bead at the droplet formation site in a microfluidic generator (17, 18). Once the droplet is formed the bead is held inside the droplet by the droplet-medium interfacial tension. Hence, it is not amenable to implement multistep solid-phase synthesis reactions on individual beads using separate reagent microdroplets for each step. To address this long-standing challenge and leverage the immense potential advantages of droplet microfluidics in solid-phase DNA synthesis (12,19), the present SBDSPS approach encapsulates and ejects the bead from the reagent droplet after its formation (FIG. 1A). It employs active dielectrophoretic (20-24) manipulation to overcome the capillary forces and move the bead across the reagent-oil interface; thus, opening an avenue for the manipulation of microparticles across immiscible fluidic interfaces. A key experimental finding which is verified through detailed numerical modeling is that the droplet does not break-up into smaller fragments in the process. This is vital to prevent contamination of reactions and spurious detection signals. The dielectrophoretic force ensures precise spatial alignment of the solid support (bead) and the reagent droplet, a factor that eludes microarrays. As a proof-of-concept, demonstrated herein is enzymatic coupling of fluorescently labelled bases to the 3′ end of the initiator strands on the bead using SBDSPS. Furthermore, it is shown using fluorescence measurements that by improving the bead surface-to-reagent ratio SBDSPS achieves higher fidelity of reactions than traditional benchtop platforms with stacked beads. The improved fidelity using SBDSPS will potentially pave the way for the synthesis of longer oligomers with considerably higher yield per bead (FLP, fewer errors per bead).

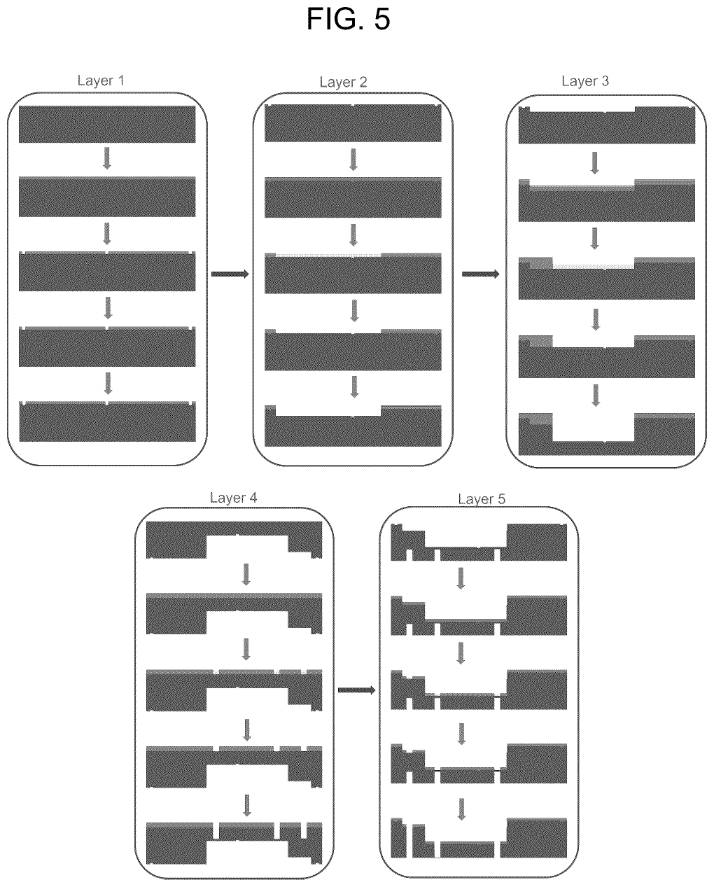

The present device is a silicon-on-glass microfluidic device with an on-demand droplet generation channel (25) and Indium Tin Oxide (ITO) electrodes (FIG. 1B-C). The component dimensions are judiciously designed (FIG. 1C). The channel 25 μm wide) dispenses a single droplet 50 μm diameter) on-demand into the reaction chamber. The electrodes in the chamber are suitably aligned with the droplet generation channel to ensure that the generated droplet lies within their trapping range (FIG. 1B-C). They serve dual purpose of spatially localizing the bead and the droplet by dielectrophoretic trapping, as well as encapsulating and ejecting the bead from the droplet through electric field-induced change in the equilibrium bead-droplet configuration. The electrodes 15 μm) are made smaller than the channel 25 μm wide) (FIG. 1C) to ensure that the trapped droplet encapsulates the trapped bead as it covers the electrode (FIG. 1A). ITO and glass ensure optical transparency for imaging the trapping and manipulation process as well as fluorescence detection of the coupling reaction on an inverted microscope. Ports for external electrical and fluidic connections are provided on the back of the device (FIG. 1C, refer to FIG. 5-11 for detailed fabrication process).

6 μm diameter streptavidin-coated fluorescent green polystyrene beads (FIG. 1D) were used as solid supports. A 5′ biotinylated oligonucleotide with 25 bases (T25mer) is used as the initiator strand, which is tethered to the microbead using a streptavidin-biotin linkage (FIG. 1D(i)). Coupling of the fluorescently labelled base (dCTP-AF647) to the 3′ end of the initiator is enabled by the enzyme terminal deoxynucleotidyl transferase (TdT) (FIG. 1D(ii)). The long initiators (>20 bases) minimize steric hindrance on TdT due to the bead surface (14). Here, the reagents consisting of dCTP-AF647 and TdT in a buffer solution (SI) form the droplet phase in the microfluidic device (FIG. 1A). A tiny amount of sodium fluorescein is added to the reagent to trace its flow through the device while ensuring ample brightness contrast between the bead and the droplet to clearly record the engulfing and ejection process using a blue LED. A red LED excites the fluorophore AF647 to detect the coupling of dCTP-AF647 to the T25mer (FIGS. 1A and 1D(ii), refer to Experimental section below for detailed sample preparation and experimental setup).

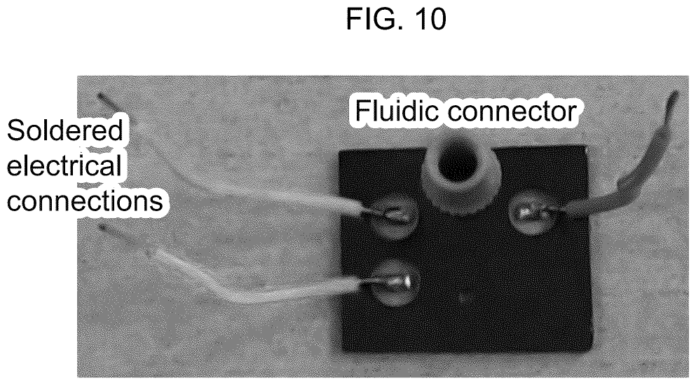

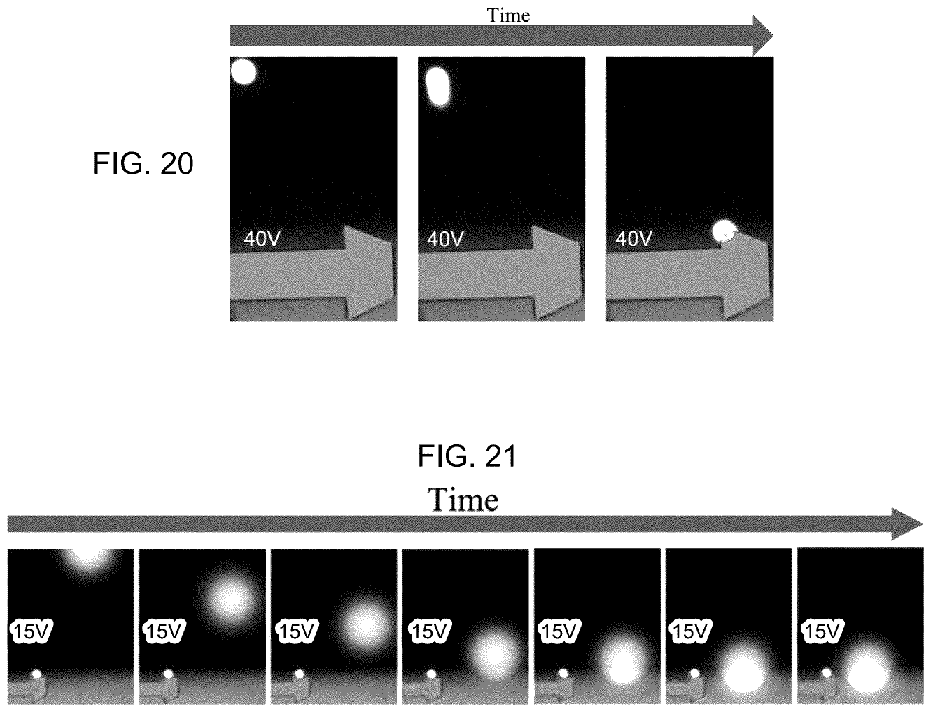

The device, filled with 2.5% w/w solution of Span 80 in silicone oil (kinematic viscosity=1 cSt), is mounted on the sample holder with relevant electrical and fluidic connections. Beads with initiator strands suspended in the oil solution are introduced into the device through the bottom fluidic port (FIG. 1C). The bead concentration is tuned to ensure a single bead enters the vicinity of the electrodes and is trapped dielectrophoretically (FIG. 20) by applying a voltage (Vₛ) across the electrodes (E₁ and GND in FIG. 10). Subsequently, a reagent droplet is dispensed into the reaction chamber and trapped adjacent to the bead (FIG. 21).

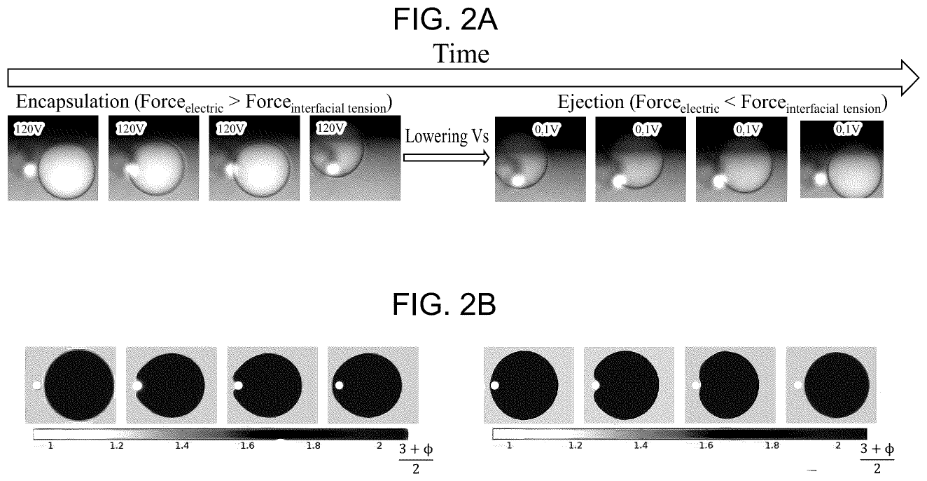

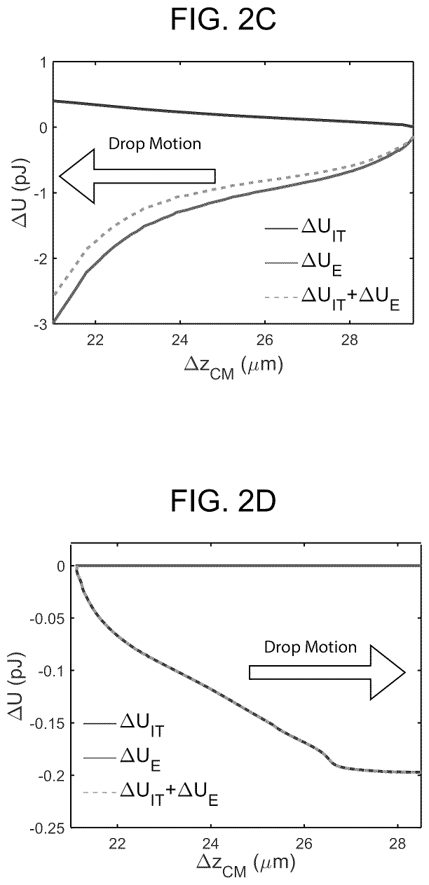

Increasing Vₛ between the electrodes to 120 V (amplitude), the bead is fully encapsulated by the reagent droplet (FIG. 2A). Reducing Vₛ to 0.1 V ejects the bead from the droplet (FIG. 2A). The observations agree with the electrohydrodynamic simulations for a simplified axis-symmetric system (FIG. 2B) and can be understood in terms of its electrocapillary potential energy (U) (26). The change in the potential energy (ΔU) of the system is due to a change in the Gibbs free energy (ΔU_{IT}≈γ_{ow}A_{ws} cos(θ)) of the interfaces between the bead, droplet and the medium as well as the change in the electric energy (ΔU_{E}=−Δ(QVₛ)/2) stored in the system. Here, γ_{ow}=5.5 mN/m is the interfacial tension between the reagent droplet and the silicone oil suspension medium, A_{ws} is the area of the bead covered by the aqueous reagent droplet, and θ=150° is the contact angle that the reagent droplet forms on the surface of the bead and Q is the charge stored on the gradient field electrode. The reduced Y_{ow} due to the added surfactant leads to the large 0. Therefore, the bead has a propensity to remain in the oil medium. When (Vₛ) is high, |ΔU_{E}|>>|ΔU_{IT}|. To attain the minimum energy configuration, the droplet moves towards the electrode (ΔU≈ΔU_{E}<0) and engulfs the bead in the process (FIG. 2C). When Vₛ is low, |ΔU_{E}|<<|ΔU_{IT}|. To attain the minimum energy configuration, the droplet ejects the hydrophobic bead, which is touching the droplet-medium interface from the inside, as it moves away from the electrode (ΔU≈ΔU_{IT}<0, FIG. 2D). Once the bead completely separates from the droplet ΔU_{IT} is negligible (FIG. 2D). While a larger droplet would mean more reagent usage per reaction a smaller droplet would require a larger voltage to exert significant dielectrophoretic force (ΔU_{E}∂Volume and ΔU_{E}∂|Vₛ|²) to move the bead across the interface. Silicone oil acts as a chemically inert suspension medium with low viscosity and high dielectric breakdown strength that prevents the rapid evaporation of the tiny reagent microdroplets due to their high surface-to-volume ratio (27). Therefore, a systematic approach to encapsulate and eject individual beads from droplets was established.

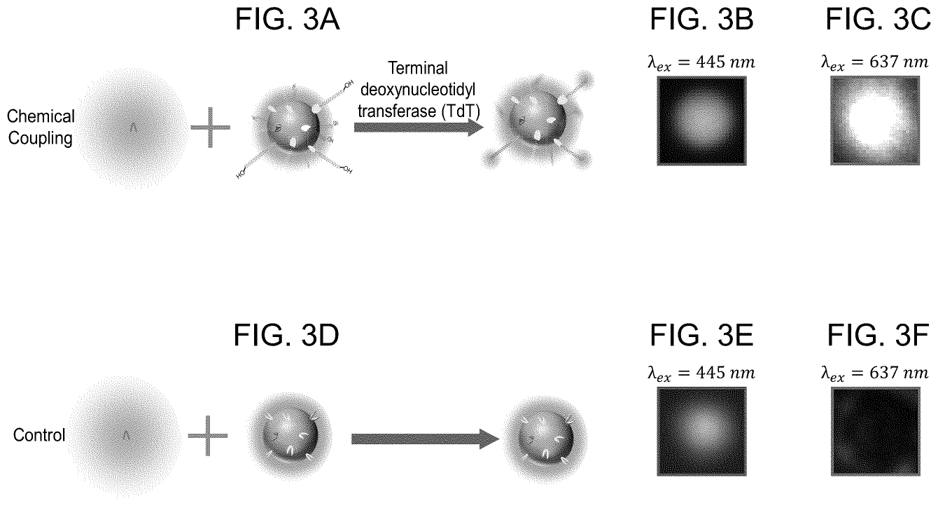

After establishing the fundamental physical working principle of SBDSPS, single bases were enzymatically coupled onto the initiator strands bound to a microbead. The above process was duplicated followed by fluorescent imaging with a red LED, which showed a clear red emission at the position of the bead (FIG. 3A-C). This confirmed the attachment of the fluorescently tagged base (dCTP-AF647) to the bead. To eliminate any false positives due to unincorporated bases non-specifically bound to the surface of the bead, the above process was reiterated using beads devoid of initiator strands and reagent solution without TdT (control experiment in FIG. 3D). Absence of non-specific binding was confirmed by the lack of red fluorescence at the site of the bead (FIG. 3E-F) after ejection from the reagent droplet. This establishes SBDSPS as a robust approach for enzymatic DNA synthesis on individual beads in droplet reactors. While prior reports of chemical synthesis in microdroplets involved all the reagents in fluidic form (28, 29), there was no droplet microfluidic analogue to solid phase synthesis owing to the lack of a physical approach to handle solid supports in such systems (12, 19). The present work brings together the disparate fields of droplet microfluidics and solid-phase synthesis.

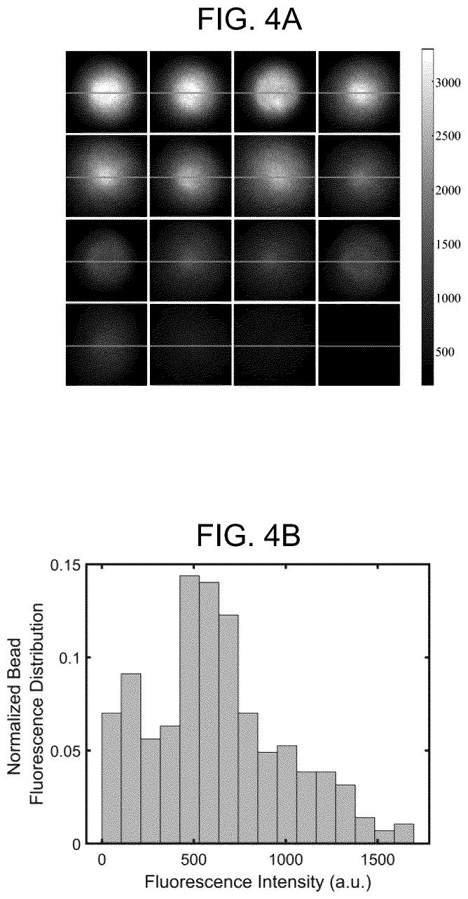

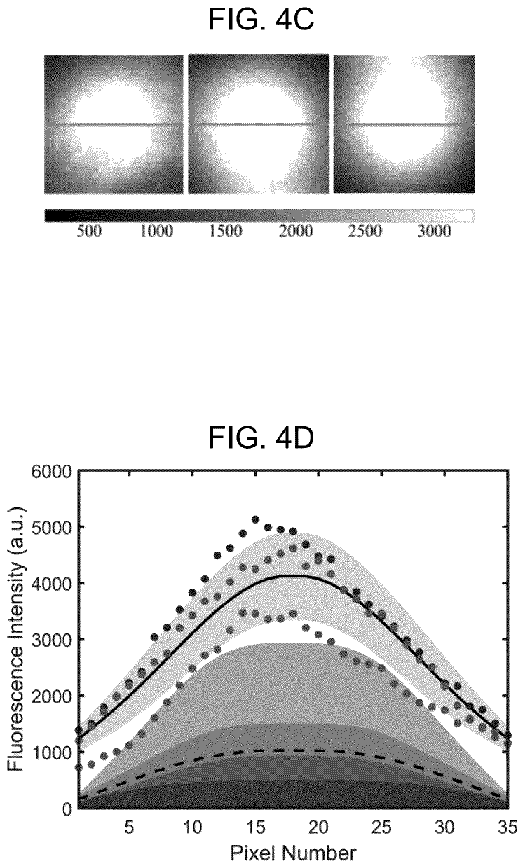

When beads from the benchtop reaction were analyzed (bulk synthesis of 1000s of beads pooled in a single 1.5 ml Eppendorf tube), large variations in fluorescence intensity across beads was found (FIG. 4A). This indicates nonuniform reagent environment in the vicinity of each bead due to bead-bead stacking. The brightest bead (top left corner of FIG. 4A) closely represents the maximum attainable base coupling. However, only a small fraction of the beads reacted on the benchtop exhibit such high fidelity (FIGS. 4A and B). Bead-bead stacking curtails the bead surface-to-reagent ratio on overwhelming majority of the beads which leads to an overall decrease in the average coupling efficiency. Thus, by eliminating bead-bead stacking and in turn beads with poor reaction fidelity, SBDSPS can potentially yield higher coupling efficiencies on an average than the traditional benchtop process. This is confirmed by the three reacted beads using SBDSPS in FIG. 4C whose fluorescence intensities (3 scatter plots in FIG. 4D) are significantly higher than the beads reacted on the benchtop (bottom grey band with graded brightness in FIG. 4D). The average fluorescence of beads reacted using SBDSPS (solid black curve in FIG. 4D) is nearly thrice the benchtop average (dashed black curve in FIG. 4D) which suggests a corresponding improvement in bead surface-to-reagent ratio and reaction fidelity. Additionally, the small picolitre volume of the droplet minimizes reagent consumption and waste. Hence, SBDSPS can potentially be a rational approach for high-fidelity solid-phase synthesis with minimum reagent usage.

In summary, the present work introduces a novel technique (SBDSPS) for implementing enzymatic oligonucleotide synthesis on individual beads by combining dielectrophoresis and droplet microfluidics. Fluorescence measurements suggest that fidelity of reactions using SBDSPS can be considerably higher than bulk synthesis on the benchtop as it addresses the critical physical problem of suboptimal bead surface-to-reagent ratio that plagues benchtop platforms with many beads stacked together. The improved reaction fidelity when replicated over multiple reaction cycles can potentially enable the synthesis of ultra-long strands of oligos (>>300 bases) with unprecedent yields of the intended FLP. This will drastically reduce reagent consumption, subsequent purification and error correction requirements and the cost of synthesis per base. Furthermore, the present work expands the functionalities of droplet-based chemical synthesis systems by adding solid-phase synthesis (which is of immense practical importance) to its fold. It also enhances the repertoire of trapping and manipulation techniques by enabling passage of particles across immiscible fluidic interfaces.

The parallelizability of droplet microfluidics (25) together with particle manipulation (30) can be employed to scale up the system. This will facilitate sequential implementation of multistep synthesis cycles where 1000s of beads (each in a separate droplet reactor) are addressed simultaneously for high-throughput synthesis of longer oligos; here every bead has an optimal bead-to-reagent ratio during the entire synthesis run.

In addition, water-in-oil droplet is an excellent model system to study enzymatic reactions within volume-confined environments (31). Typically, droplets are merged to deliver the reaction substrate to the enzyme. However, this process is accompanied by an increase in the overall volume. Instead, if the substrate is bound to a solid support, as in SBDSPS, the volume of the aqueous compartment remains the same. Digital microfluidics has enabled the processing of chemical information using Boolean logic, paving the way for chemical computation as well as logic-driven manipulation of chemical payloads for combinatorial screening applications (32). SBDSPS can extend this concept a step further by writing chemical information onto solid supports; thus, creating solid-state drives for chemical information, which can facilitate one-bead-one-compound combinatorial synthesis (33) using Boolean logic as well as DNA data storage (6,7) on a digital microfluidic processor. SBDSPS can be extended to implement other forms of solid-phase synthetic chemistries such as oligopeptide, and oligosaccharide (14). Finally, the diminishing costs of performing solid-phase combinatorial chemistry using the one-bead-one-compound (33) approach on SBDSPS is expected to make it economically feasible for a larger body of scientists to pursue research into traditionally expensive areas of chemistry such as drug discovery and vaccine development, which in turn is expected to expedite scientific discoveries to cure potentially life-threatening diseases and ailments.

### Materials and Methods

Device Fabrication

Device fabrication started with ˜500 μm thick silicon wafers (p-type, 10-20 Ωcm, <100>) and borofloat glass wafers ˜550 μm thick that are 100 mm in diameter.

The silicon wafer was etched in 5 layers to define the alignment marks, microfluidic channel for droplet generation, reaction chamber and ports for external fluidic and electrical connections. In the 1^{st} layer the alignment marks (crosses that are 300 μm long and 10 μm thick) were etched to a depth of 1 μm using reactive ion etching after a standard photolithography process. These alignment marks would facilitate proper alignment of subsequent layers as well as the alignment and bonding of the silicon wafer and the borofloat glass wafer. In the 2^{nd} layer, the microfluidic channel, chamber, and the fluidic ports were etched to a depth of 25 μm using reactive ion etching. This defines the height of the microfluidic droplet generator channel. In the 3^{rd} layer, the chamber and the port were etched to an additional depth of 175 μm using reactive ion etching. This sets the height of the reaction chamber at 200 μm (25 μm+175 μm). The 4^{th} layer involves etching almost through the ports by etching a further depth of 200 μm from the back side of the wafer using reactive ion etching. In the 5^{th} layer, the ports were completed by laser drilling through the ports from the front side. The etching of the ports was done in two layers as etching through the wafer using a reactive ion tool would require additional support wafer on the back side to prevent the sample holder from being exposed to the plasma. The support wafer is generally attached using photoresist or oil. Any nonuniformity in the spread of the photoresist/oil would lead to a corresponding nonuniformity in contact between the device wafer and the support wafer. This will cause significantly varying heating effects and etch rates across the wafer which may lead to unwanted damage to the device features. The high heat produced during RIE will lead to strong baking of the resist making it difficult to strip the resist of the small device features (in case photoresist is used to attach the wafers). SF₆ gas was used for the reactive ion etching of silicon in each step. The photoresist for each layer is patterned using a standard photolithographic approach using a maskless exposure system (Heidelberg MLA 150). Post etching of each layer the photoresist is stripped off from the wafers using oxygen plasma and cleaned for the next layer in piranha (9:1::H₂SO₄:H₂O₂) at 120° C. for 20 minutes. The step-by-step schematic of the fabrication process of the silicon wafer is shown in FIG. 5.

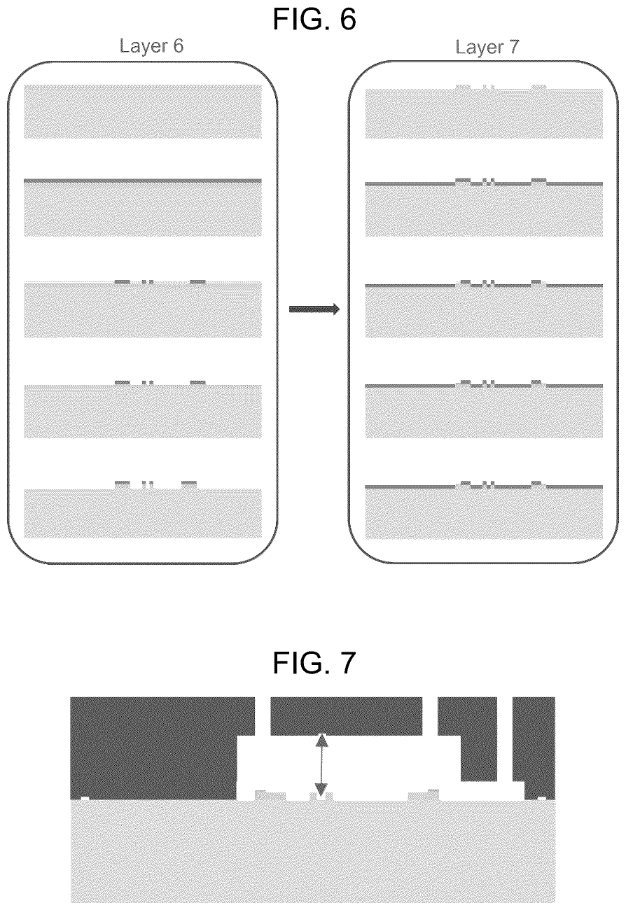

The borofloat glass wafers were piranha (9:1::H₂SO₄:H₂O₂) cleaned at 120° C. for 20 minutes following which 800 nm of ITO (Indium Tin Oxide) was sputter deposited on it at LGA Thin Films. This was followed by 2 layers of photolithographic processing to define the ITO electrodes and the gold contact pads. In the 1^{st} layer the electrodes and alignment marks were defined by reactive ion etching of ITO from the entire wafer (barring the electrodes) using CH₄ and H₂ gases. The alignment marks would be used for alignment of subsequent layers in the glass wafer processing as well as alignment with the patterns on the silicon wafer while wafer bonding. In the 2^{nd} layer, the contact pads were defined by depositing 10 nm Cr and 200 nm Gold evaporatively followed by a metal liftoff process. A standard photolithographic process was followed to pattern the photoresist for etching and deposition in the above layers. After every layer, the wafers were sonicated in acetone followed by oxygen plasma cleaning to strip the resists and any other organic material. This was followed by wet cleaning in 5:1:1::H₂O:H₂O₂:NH₄OH at 70° C. for 1 hour. The step-by-step schematic of the fabrication process of the borofloat glass wafer is shown in FIG. 6.

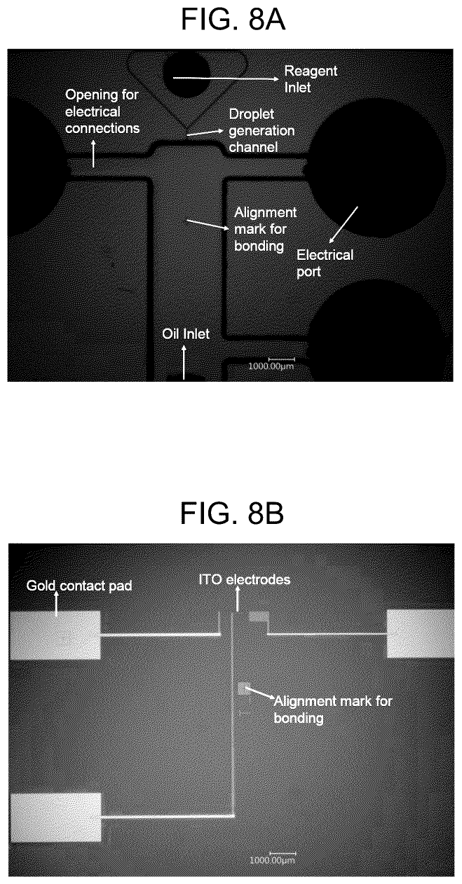

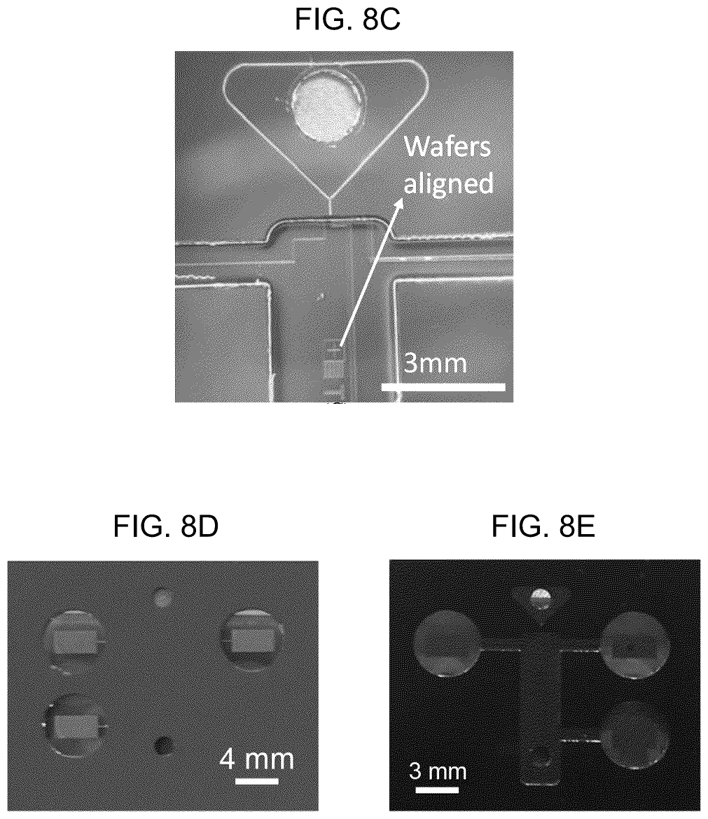

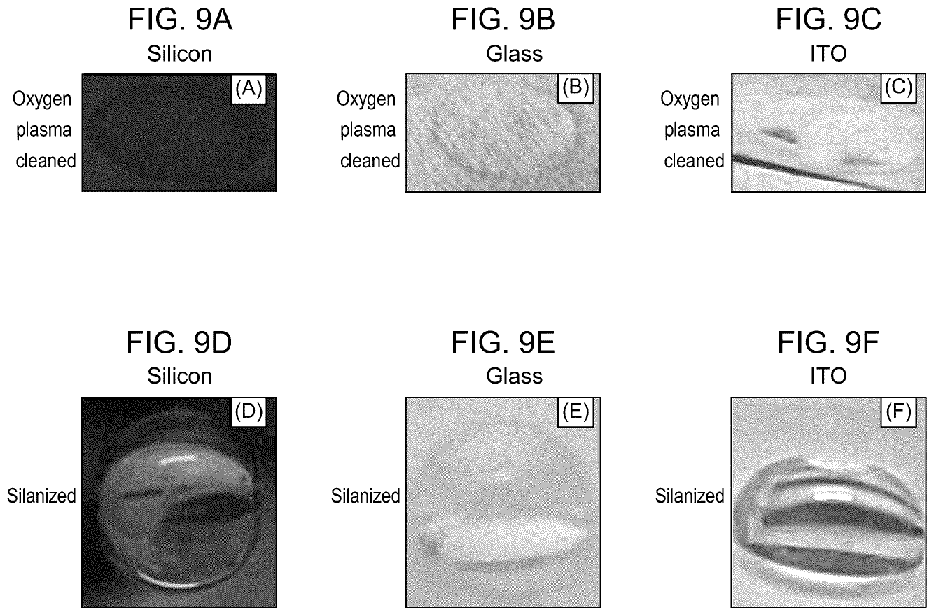

The glass and silicon wafers are then aligned and bonded anodically at 350° C. by applying a voltage of 350V for 6 mins (FIG. 7). The wafers are then diced into 25 mm×35 mm chips using a laser cutter. The optical micrographs of the fabricated silicon structure, glass structure, the aligned wafers and the final device are shown in FIG. 8. The chips are cleaned in oxygen plasma and then coated with Dimethyldichlorosilane (DDMS) using vapor phase deposition at Integrated Surface Technologies. Silanization makes the device interior surfaces hydrophobic enabling the formation of water droplets (Native oxide formation on exposed silicon surfaces makes them hydrophilic. Glass is chemically silicon dioxide and hence it is hydrophilic. Without silane coating, the aqueous phase will wet the device surface and prevent the formation of water droplets). Demonstrated herein is the effect of silanizing silicon, glass, and ITO surface (FIG. 9). The silane on the exterior surface of the chip prevents adhesion of glues necessary to stick fluidic connectors on the chip. So, it is stripped using UV ozone at 90° C. for 20 mins after covering the device ports. Fluidic connectors are then glued to the chip using Loctite 401 adhesives (FIG. 10). Electrical leads are soldered onto the gold contact pads through the electrical ports in the silicon wafer (FIG. 10).

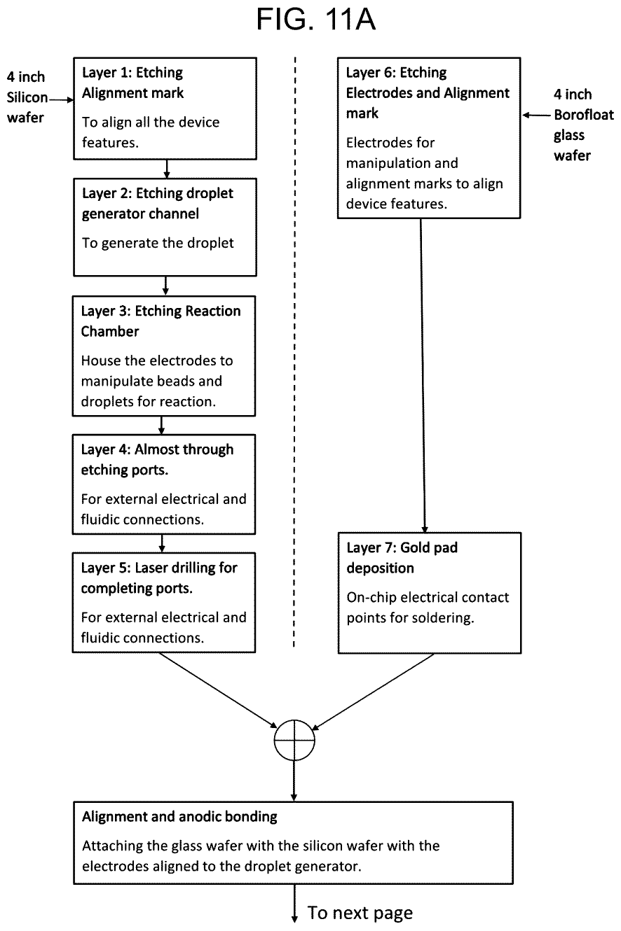

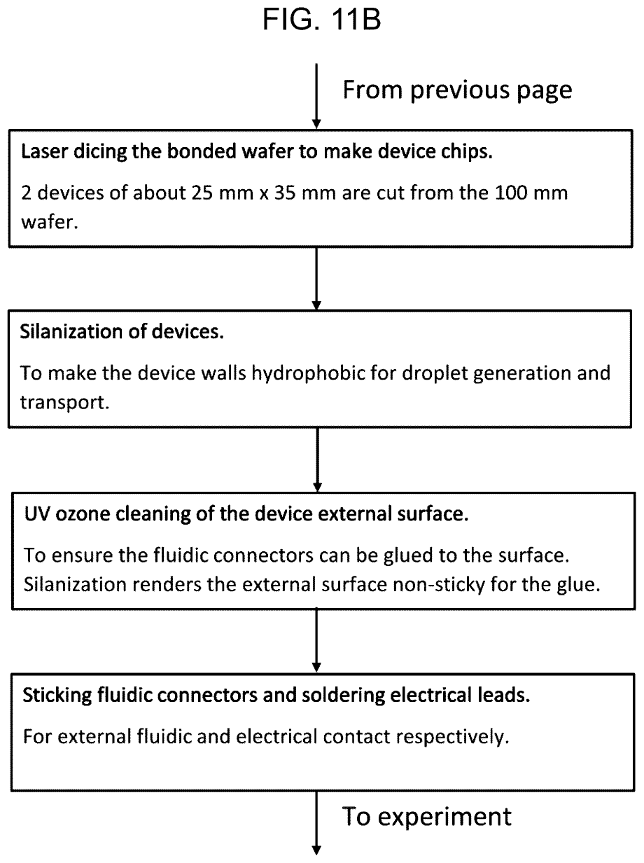

The entire fabrication process flow is summarized in FIG. 11.

Experimental Setup

The experimental setup consists of the fabricated device interfaced with the fluidic, electrical, and optical subsystems.

Device

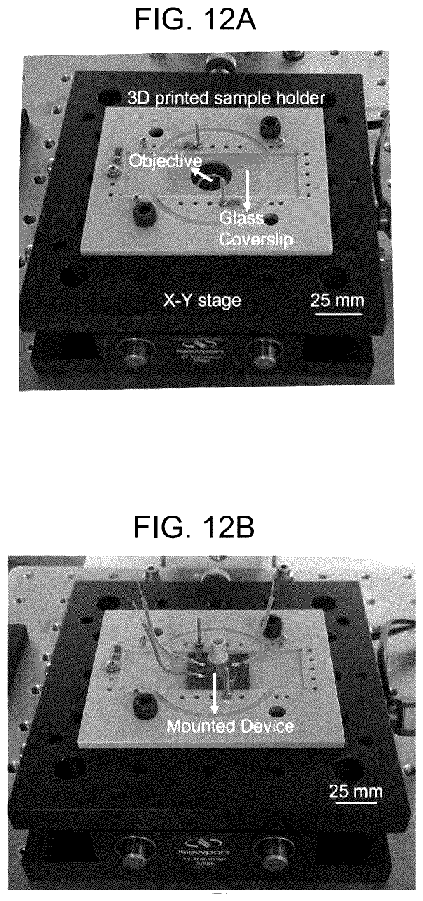

The fabricated device is mounted on a 3D printed plastic sample holder with a 0.17 mm glass coverslip (24 mm×40 mm) from SPI below it. Copper pins on the sample holder hold the device in place. The sample holder is screwed onto a X-Y stage from Newport (Model #-406) mounted on a modified Nikon TE-2000U inverted microscope. This is shown in FIG. 12.

Fluidic Subsystem

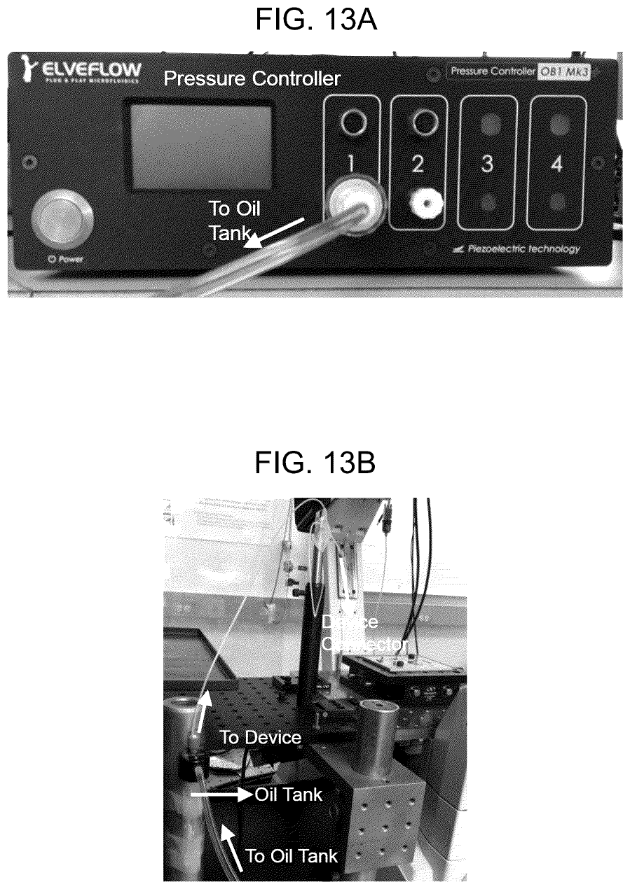

The fluidic subsystem (FIG. 13) consists of piezo driven pressure controller (OB1 MK3+ from Elveflow) which is provided with an input from the house nitrogen supply at 30 psi. The output, which can be a maximum of 2000 mbar, is connected to the input of a fluidic tank which is a 15 ml plastic tube using a 10 mm OD tubing. The output of the tank flows into the device via a 1/16-inch OD and 1/32-inch ID polytetrafluoroethylene (PTFE) tubing from Masterflex (item #-EW06407-41) which is plugged into the fluidic port of the device using the Nanoport Assemby for 1/16 inch OD tubing from IDEX Health & Science (part #-N-333). The height of the tank is adjusted using a post to ensure that the supply pressure needed to drive fluid flow inside the microfluidic channel is within the range of the pressure controller. The pressure input and the fluidic output to the tank (which is the 15 ml plastic tube) are connected using a custom designed cap cum connector. The fast switching of the piezo driven pressure controller allows for the generation of a single droplet only.

Electrical Subsystem

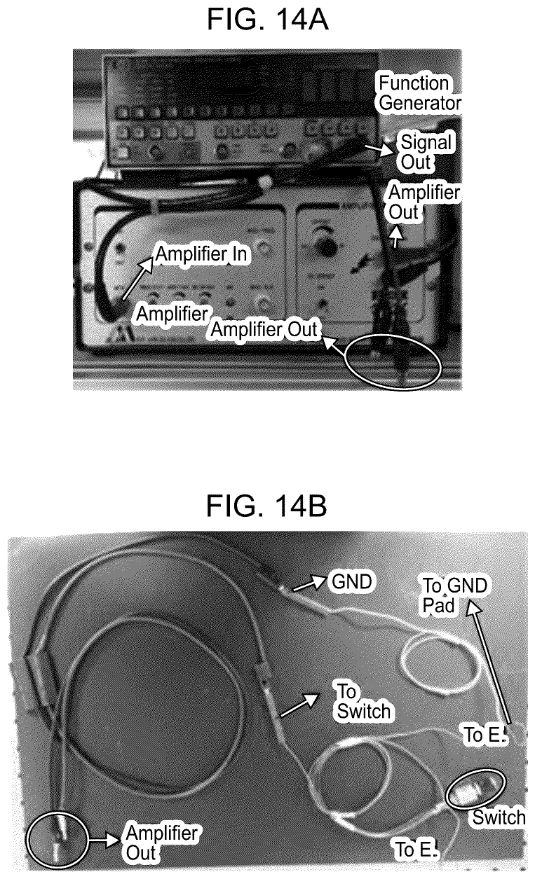

The electrical subsystem (FIG. 14) consists of a function generator from Hewlett Packard (Model #-8116A) whose output is amplified by a A.C. high voltage amplifier from A. A. Lab Systems Ltd. (Model #-A-303). A maximum input amplitude of 10 V (amplitude) can be supplied to the amplifier, and it can generate a maximum output amplitude of 200 V (20× amplification). The amplified output is connected to the electrical leads of the device through a single pole double throw (SPDT) switch which connects the supply across either of the electrodes (E₁ or E₂) and the ground pad. The output of the function generator is fed to the input of the amplifier using a BNC cable. The output of the amplifier is fed to the device using banana cables through the SPDT switch. 125 mA fuses are inserted in the output circuit of the amplifier to prevent damage to it due to excess current.

Optical Subsystem

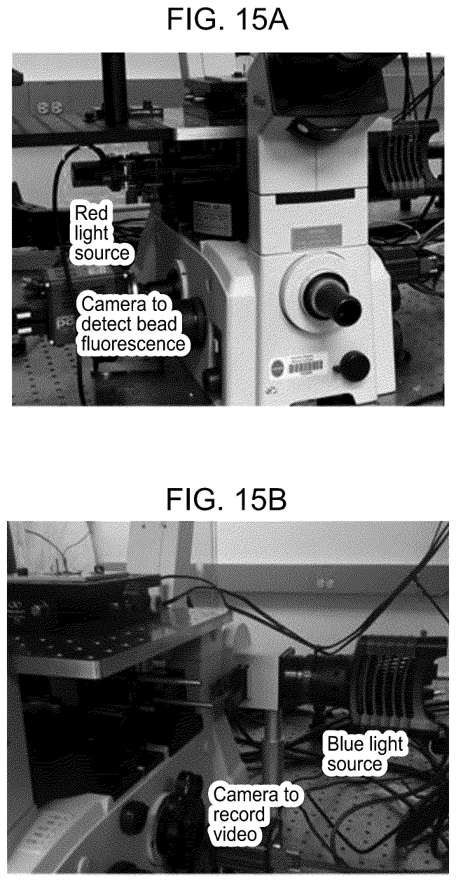

The optical subsystem (FIG. 15) is built around an inverted TE-2000U microscope from Nikon which has been appropriately modified. The imaging system consists of two sources. A blue LED (SOLIS-445C from Thorlabs, 445 nm and 5.4 W min) with a band pass excitation filter (D480/30x from Chroma) images the bead (6 μm diameter fluorescent green streptavidin coated polystyrene beads with excitation maxima at 441 nm and emission maxima at 486 nm, Catalog #-24157) and the fluid flow in the device through their green fluorescence emission onto a sCMOS camera from Thorlabs (Part #-CS2100M-USB). The experiments are recorded at 33 frames per second. A red LED (M625L4 from Thorlabs, 625 nm and 700 mW) is used to excite the fluorescence from Alexa-647 which is used as a fluorescence label attached to the nucleotides (dCTP) in the reagent droplet. A sCMOS camera from PCO (PCO edge 5.5) is used to image the low light intensity levels emanating from the nucleotides coupled to the initiator strands on the beads. The fluorescence is captured with a 2 s integration time. Appropriate bandpass excitation (Item #-86-988 from Edmund Optics, 640 nm center wavelength, 14 nm bandwidth, OD—6) and emission (Item #-86-987 from Edmund Optics, 676 nm center wavelength, 29 nm bandwidth, optical density—6) filters are used to ensure non-overlap of the emission spectrum of the red LED and that of Alexa-647. A Nikon objective (ELWD-20) with 20× mag and 0.45 NA is used. The objective has a correction collar for spherical aberration correction. This collar is set at 0.7 mm (0.17 mm thick glass coverslip+0.53 mm thick borofloat glass of the device).

Sample Preparation

Preparing the Oil Solution by Adding Surfactant

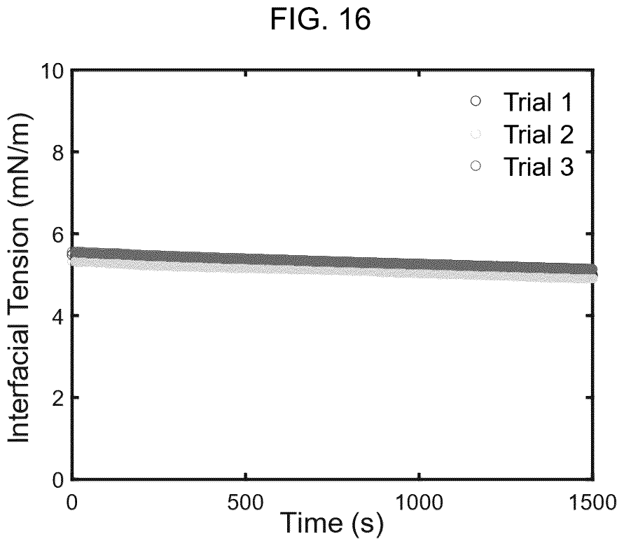

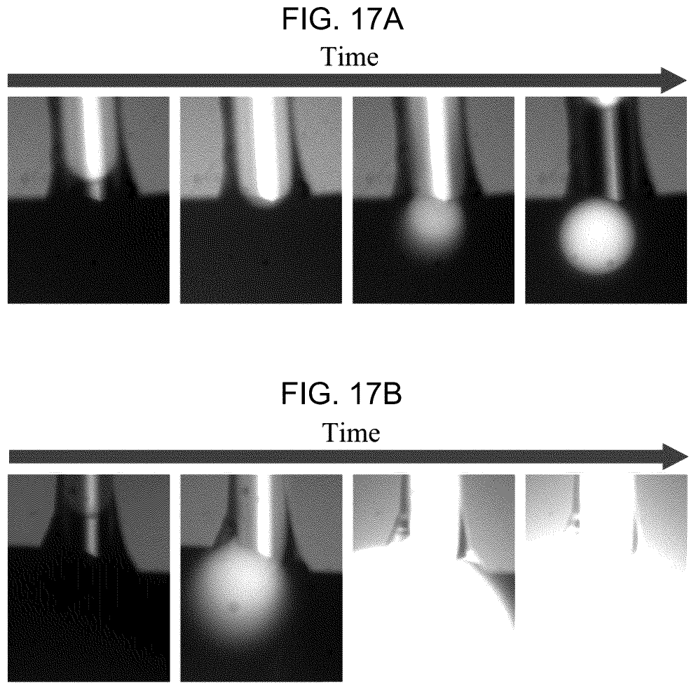

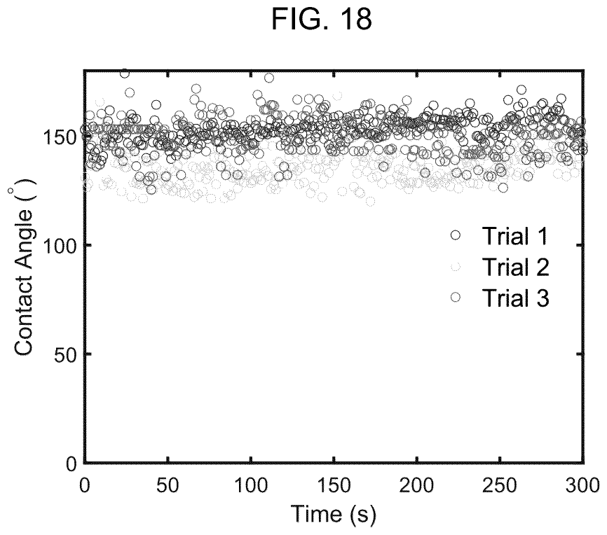

The continuous medium in the microfluidic device is 1 cSt silicone oil (PSF—1 cSt from Clearco Products) with 2.5% w/w Span 80 (S6760 from Sigma Aldrich). This was prepared by adding 4 ml of Span 80 in 200 ml silicone oil and sonicating it for 30 minutes to ensure the surfactant completely dissolves in the oil. Silicone oil acts as a low viscosity suspension medium for ease of movement of the beads and droplets under the dielectrophoretic force. It has a high dielectric breakdown field strength which allows the usage of high applied voltages to encapsulate and eject beads from droplets. It also prevents the rapid evaporation of picoliter droplets. The addition of surfactant reduces the interfacial tension between water and silicone oil from 36 mN/m (34) to 5.5 mN/m (FIG. 16). It serves multiple purposes:
The reduction of the interfacial tension between the reagent and oil medium also helps increase the contact angle the reagent forms with the silanized walls of the device. The reduced interfacial tension and the larger contact angle facilitates droplet generation (FIG. 17A). In the absence of the surfactant droplet breakup does not take place (FIG. 17B).
This reduced interfacial tension allows overcoming the interfacial tension barrier using the dielectrophoretic force to encapsulate and eject the bead from the droplet.
The reduced interfacial tension likely contributes to the large contact angle that the reagent forms on the surface of the streptavidin coated polystyrene bead which increases the tendency of the bead to seek the suspension medium over the reagent droplet (to estimate this, the contact angle that a reagent droplet forms on a streptavidin coated glass slide in a medium of silicone oil with Span80 was measured, FIG. 18). So, as soon as the electrical supply voltage is reduced, and the electrical energy of the system becomes negligibly small compared to the Gibbs free energy of the fluidic interfaces the bead spontaneously comes out of the droplet once it touches the droplet-medium interface. The smaller the contact angle the larger the tendency of the bead to stay attached to the droplet.

Attaching Initiator Strand to Beads

The initiator strand, which is a biotinylated oligomer with 25 bases (T25mer, 5′ biotin, IDT) is attached to the 6 μm diameter streptavidin coated green fluorescent polystyrene beads using the strong biotin-avidin hydrogen bond. The reaction was carried out for 60 min at 23° C., 14 RPM. The approximate yield (attomoles per bead (T25mer bound)) was determined by measuring the optical density at 260 nm (Nanodrop One C, ThermoScientific) by subtracting the supernatant and wash OD values from the starting yield. Binding/wash buffer: 20 mM Tris pH 7.5, 1 M NaCl, 1 mM EDTA, 0.0005% Triton-X 100 (45 μl (plus 5 μl 100 μM T25) for binding reaction, and 500 μl for wash steps). Based on a particle concentration of 1.25%, approximately 620,000 beads per 25 μl reaction were calculated (accounting for a 20% loss due to mixing and wash steps). These beads with initiator strands were then spun down using an Eppendorf Minispin (Catalog #-022620100) to remove the supernatant and segregated into two parts (i) for Single Bead Solid-Phase Synthesis (SBDSPS) in the fabricated chip, and (ii) for benchtop synthesis in 1.5 ml tubes containing many stacked beads. Benchtop synthesis is used for reagent optimization and optimal process development which can then be used to implement SBDSPS in the fabricated device as well as to compare coupling reaction efficiencies using SBDSPS and the bulk (benchtop).

Suspending Initiated Beads in Oil Solution

To implement enzymatic coupling using SBDSPS, the spun down beads with initiators are suspended in the oil solution by sonication. The concentration of the beads in the silicone oil solution are tuned to ensure mostly a single bead floats in the vicinity of the electrodes within the field of view of the objective.

Preparing Reagent Solution

The reagent solution is prepared by mixing 25 μl of reagents consisting of the fluorescently labelled base (dCTP-AF647) in a buffer solution of 50 mM Potassium Acetate, 20 mM Tris-acetate, 10 mM Magnesium Acetate, and 0.25 mM Cobalt Chloride with the enzyme (TdT) solution consisting of 3 μl of 50 mM KPO4, 100 mM Sodium Chloride, 1.43 mM β-ME, 50% glycerol, and 0.1% Triton X-100 solution in an Eppendorf tube. This reagent solution was formulated by initial benchtop experiments as described in the subsequent experimental procedure section. A trace amount of sodium salt of fluorescein (F6377 from Sigma Aldrich) is added to the reagent using a toothpick to discriminate it from the continuous phase inside the microfluidic device.

Filling Device with Oil Solution as Continuous Phase

50 ml of the 2.5% w/w solution of Span80 in 1 cSt silicone oil is taken in a glass jar. The device is completely immersed in it and then placed in a vacuum dessicator connected to a vacuum pump. As the dessicator is evacuated the air inside the device is drawn out as evident from the bubbles emanating from the jar. When the desiccator is refilled with air, the silicone oil solution gushes into the device to fill it completely without any trapped air bubbles.

Mounting Device on Sample Stage

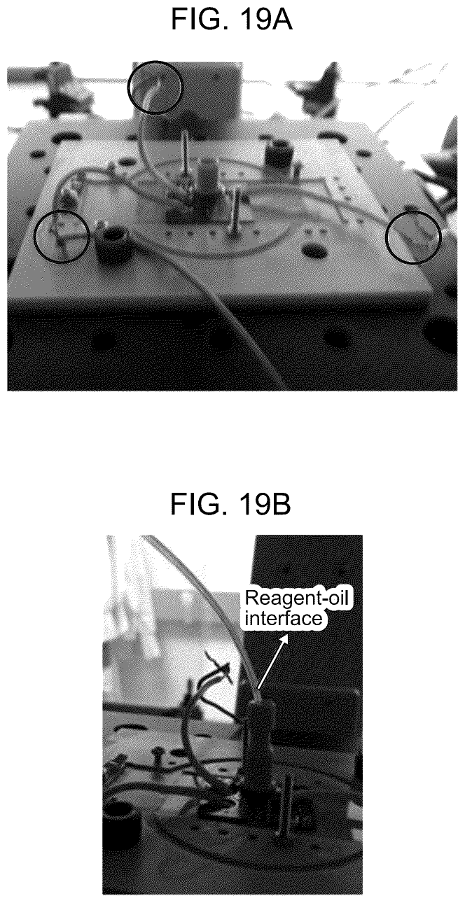

The device is then removed from the glass jar, its outer surface is cleaned by thoroughly wiping with isopropanol and then mounted on the sample holder with 24 mm×40 mm coverslip underneath which is 0.13-0.17 mm thick (FIG. 19A). The device is held firmly in place using the copper pins. The objective is focused on the output of the droplet generation channel and the ITO electrodes.

Making Electrical and Fluidic Connections to Device Ports

Electrical connections are made from the output of the amplifier to the ground pad and to the trap electrodes through the SPDT switch (FIG. 19A). The fluidic tank is filled with 15 ml of the above oil solution. A pressure is applied using the pressure controller to fill the output PTFE tubing from the tanker with oil solution which is dipped at the other end inside the 1.5 ml tube containing the reagent. Before oil starts dripping from the tubing into the 1.5 ml Eppendorf tube containing reagents, the height of the PTFE tubing and reagent tube was raised to suck the reagent into the tubing. Then the tubing was lowered again into another Eppendorf tube containing the oil solution. As the reagent solution starts dripping, the height was raised again to fill the PTFE tubing with the oil solution while ensuring there are no trapped air bubbles. At this stage the tubing is filled with oil solution at the top, the reagent in the middle and oil solution again at the bottom. The tubing is then connected into the device inlet while pushing out the oil solution at the bottom to ensure no air gaps and fluidic continuity (the oil solution inside the device and at the bottom of the tubing are the same). This approach prevents immediate flow of the reagent solution through the droplet generation channel as soon as the PTFE tubing is connected thus allowing time for experimental setup and control (FIG. 19B).

Experimental Procedure

Encapsulation and Ejection of Bead from Droplet

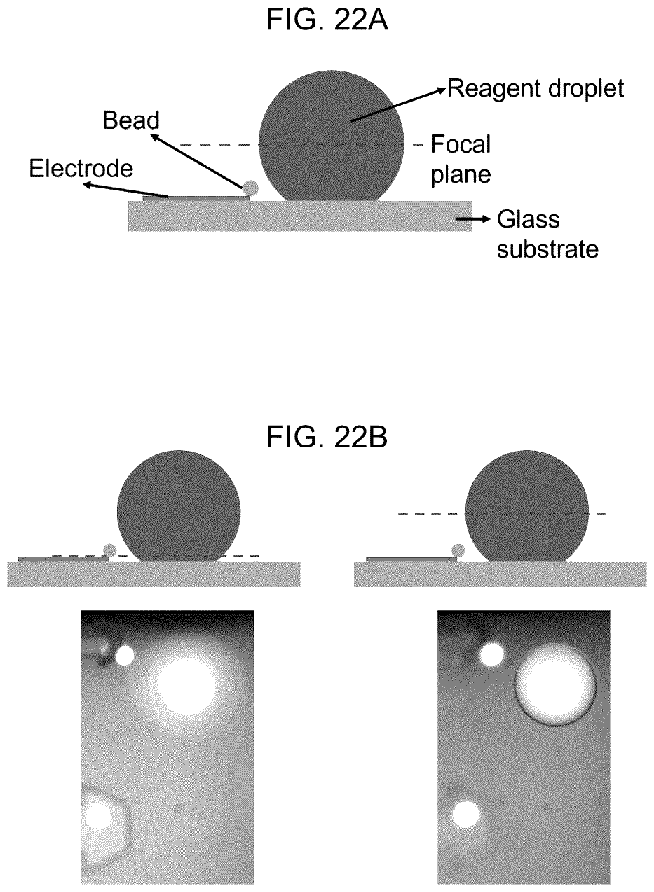

Beads suspended in the oil solution are introduced into the device through the oil inlet (FIG. 8A). Once a bead is seen near the top electrode the voltage supply is switched on and set to around 40V amplitude at 200 Hz to dielectrophoretically trap the bead (FIG. 20). After trapping the bead, the voltage supply is switched off. Following this a pressure of 60 mbar is applied on the pressure controller to push the reagent into the device. As the reagent approaches the entrance of the microfluidic channel a sudden pressure pulse of 10 mbar is exerted using the pressure controller to dispense a single droplet into the reaction chamber. Following this the voltage supply is switched on again and a voltage of 15V amplitude at 200 Hz is applied to trap the droplet adjacent to the bead on the top electrode (FIG. 21). Once trapped, the objective of the inverted microscope is adjusted to focus on the greater circle of the droplet (FIG. 22). As a result, the bead and the electrodes are somewhat defocused. This focal plane is maintained through subsequent experimental steps and all experimental data are recorded at this focal plane.

At this point the supply voltage is gradually increased to ˜120V amplitude. The droplet moves toward the electrode to encapsulate the bead. Subsequently the voltage is reduced to 0.1 V amplitude and the bead is ejected out of the droplet.

Chemical Coupling of Base and Control on the Device

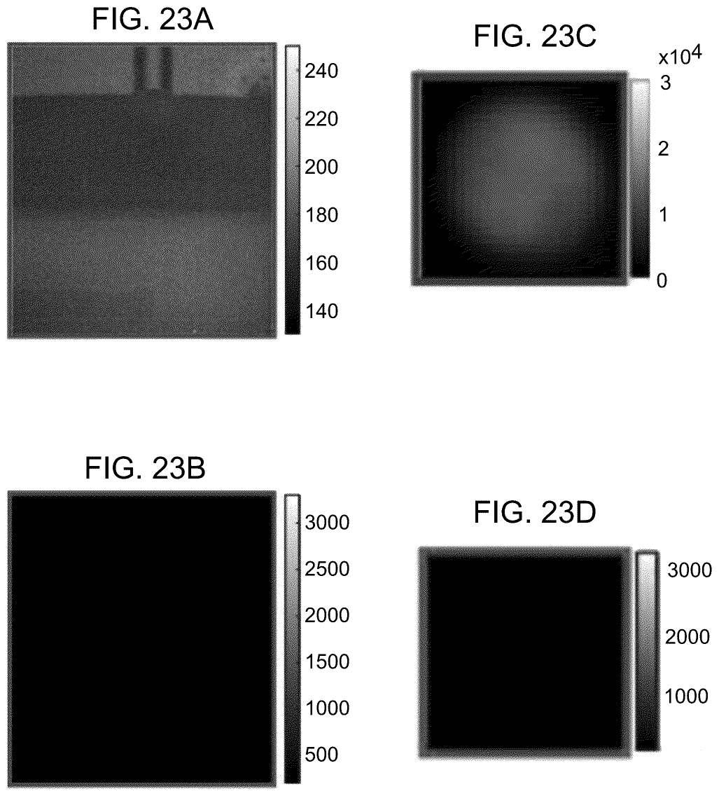

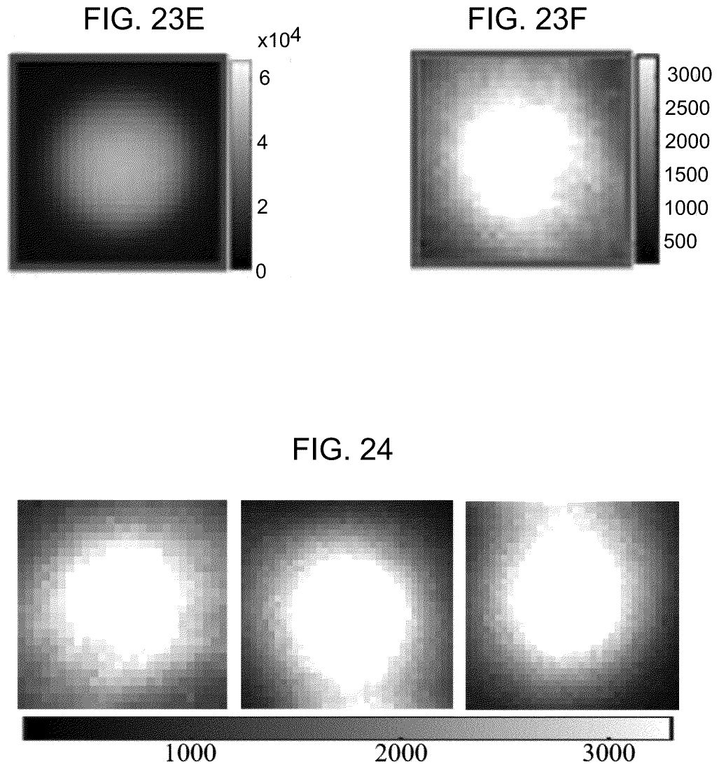

The fundamental process of bead encapsulation and ejection was used for the enzymatic coupling of a fluorescently tagged nucleotide onto the initiator strand on the bead with a few additional intermediate steps as enlisted below.
Before loading the beads into the device an image of the vicinity of the trap electrodes was captured with the red excitation using the PCO edge 5.5 camera to estimate the minimum background noise under red illumination. The fluorescence signals due to chemical coupling should be significantly larger than this for robust detection of chemical reaction (FIG. 23A-23B).
After the bead is dielectrophoretically trapped on the top electrode an image each with the blue and red excitation are captured. This is to measure the level of the red fluorescence signal from the site of the bead prior to the reaction. Any difference in the fluorescence signal after the encapsulation and ejection process can then be attributed to the attachment of the fluorophores (AF647) present in the droplet onto the bead (FIG. 23C 23D).
Finally, after the encapsulation and ejection process another set of images under blue and red excitation were captured (FIG. 23E-23F).

These images were captured as 16-bit Tiff files. FIGS. 23E and 23F are used as FIGS. 3B and C, respectively. These steps were repeated to see the repeatability of the chemical coupling reaction on the platform. The three different reacted beads are depicted separately in FIG. 24 and are also used in FIG. 4C.

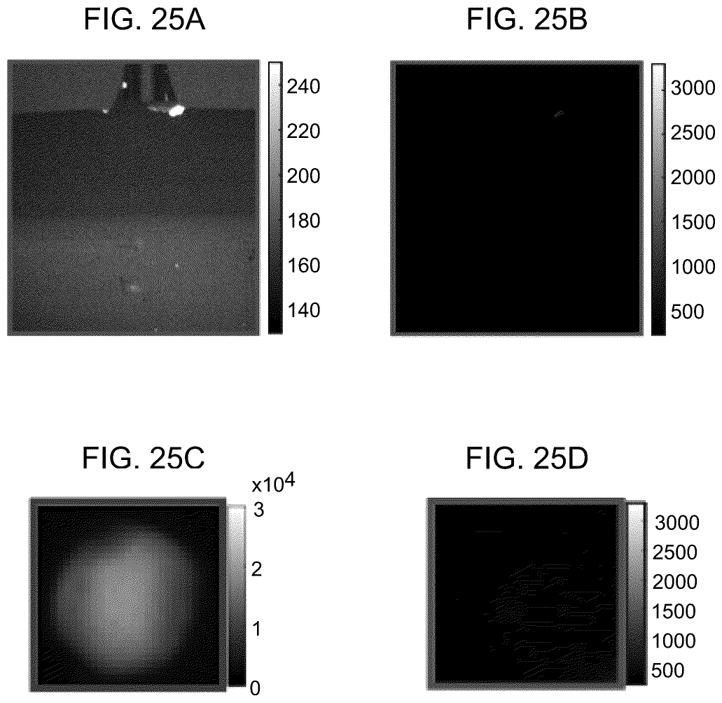

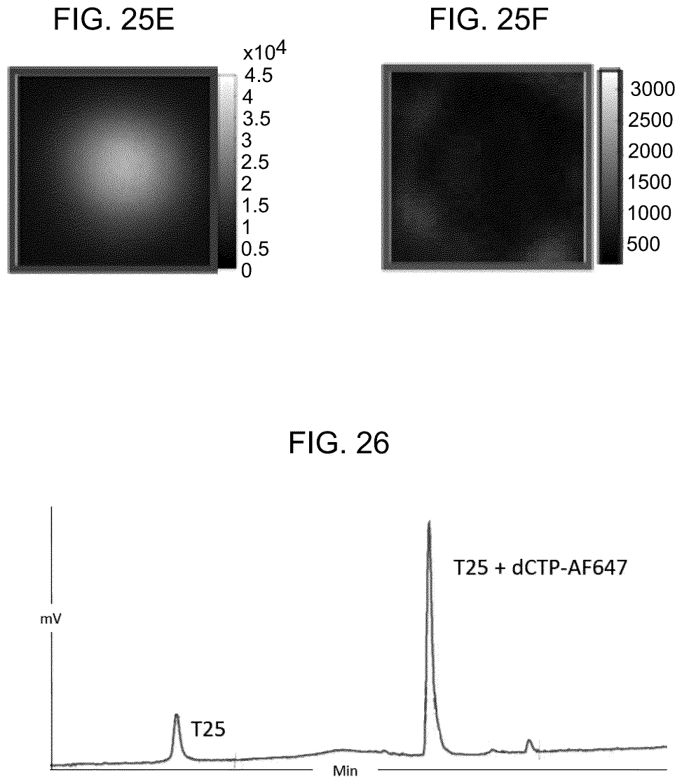

The attachment of fluorophores to the beads as in FIG. 19F can be due to (i) specific binding i.e enzymatic coupling of the fluorescently labelled dCTP to the initiator strands on the bead or (ii) non-specific binding i.e. mere physical sticking of the fluorescently tagged bases to the beads without any chemical coupling. Therefore, to establish chemical coupling and negate or eliminate the contribution of non-specific binding, the steps in the above paragraph were repeated for the control experiment which was done using beads without initiator strands and reagent droplets without the enzymes. No reaction is expected in this case. So, the absence of any red fluorescence from the bead after ejection from the droplet in this case (FIG. 25), will confirm chemical coupling in the previous case (FIGS. 23 & 24). FIGS. 25E and F are used as FIGS. 3E and F, respectively.

Benchtop Synthesis

As stated earlier, the utility of benchtop synthesis is twofold:
To develop a method/protocol of generating product on the polystyrene beads for translation onto the device. For this the reactions were performed free in solution without any solid supports.
To compare synthesis on a single bead (SBDSPS) with that in bulk (benchtop). For this the reactions were performed on solid supports (beads packed in 1.5 ml Eppendorf tubes).

Once optimized by performing free solution reactions, the benchtop reactions (25 ul) were then scaled down for performing enzymatic synthesis on the device using a single bead. Optimum conditions for enzymatically coupling dCTP-AF647 onto an initiator strand (T25) tethered to a polystyrene bead at room temperature (23° C.) (6 um dia.) are outlined in the Table below.

TABLE
Benchtop reaction setup. Either the reaction was prepared 1)
for synthesis free in-solution (no beads) where 1 μl (50
μM* strand (T25) was added to the reaction mix or 2) for
synthesis directly onto the beads (in this case 7 μl beads with
the initiator already attached were used). Buffer/enzyme (TdT)
mix: 50 mM potassium acetate, 20 mM Tris-acetate, 10 mM
magnesium acetate, 2.5 mM CoCl₂, water, pH

| Reagent | μl |
|---|---|
| TdT (20 U/μl) | 3.0 |
| Buffer (10×) | 2.5 |
| CoCl₂ (2.5 mM 10×) | 2.5 |
| Beads (7 μl) or initiator (50 | 0 (or 1) |
| μM*) | |
| Dye (1 mM) | 0.1 |
| Water* | 16.9 (or 15.9) |
| Total | 25 |

For enzymatic synthesis performed free in-solution, results were analyzed using reverse-phase high performance liquid chromatography (HPLC) (FIG. 26).

Seven μl bead solution (after initial binding) was spun down in a microcentrifuge for 30 sec, and supernatant was removed; the reagents for the synthesis reaction were then added to the beads and mixed. Samples were left at room temperature (23° C.) for 5 min, 14 RPM; post-reaction, 1 ul 10M EDTA (Ethylenediaminetetraacetic acid) was added to stop the reaction (EDTA acts as cation chelating agent to kill enzyme activity). The samples were then spun down and the supernatant was removed, followed by 3+ wash steps (until absorbance was zero) using binding buffer (from bead hydrogen bonding with initiator). The sample was then resuspended in the same binding buffer (25 μl).

As a control, synthesis was performed on beads without initiators and reagents without TdT and the reaction was analyzed through fluorescence measurements.

Measuring Fluorescence from Beads Reacted on the Benchtop in Eppendorf Tubes

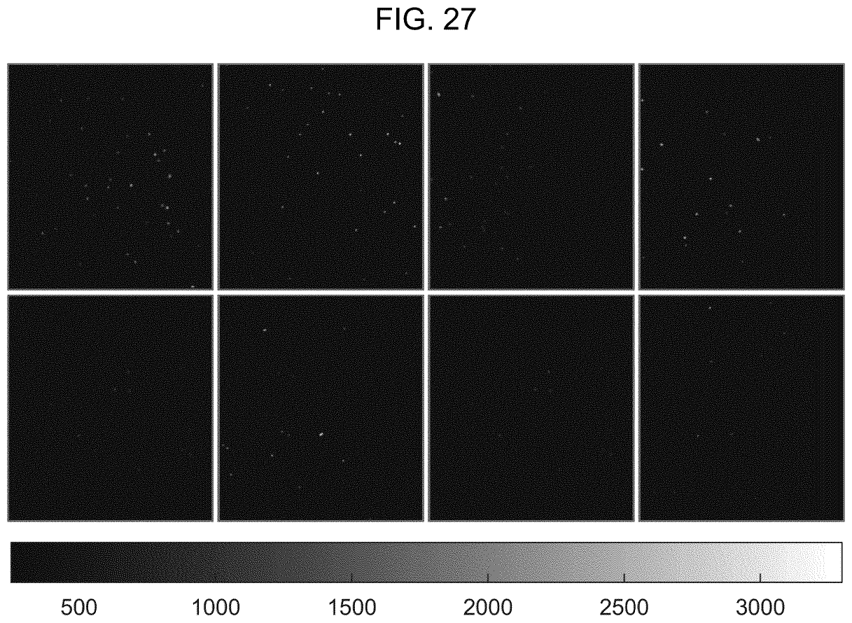

About 3 μl of the bead suspension in the buffer was taken in an Eppendorf tube and was diluted to ensure the bead concentration is small enough to prevent signal interference from beads in different planes while being large enough to have ample beads within the field of view to get a statistically significant inference about fluorescence intensity distribution. The beads were introduced into the device filled with MilliQ water. The same chips are used for the fluorescence measurements to ensure identical optical environment for comparison between on-chip experiments with their benchtop counterparts. Once the beads settle down (imaged using blue excitation), the excitation was switched to red and the fluorescence intensity of the beads was imaged. 8 frames with an average of 40 beads in each frame were captured and the fluorescence intensity distribution was observed (FIG. 27). Select beads from FIG. 27 representing the entire range of bead intensity distribution are used in FIG. 4A.

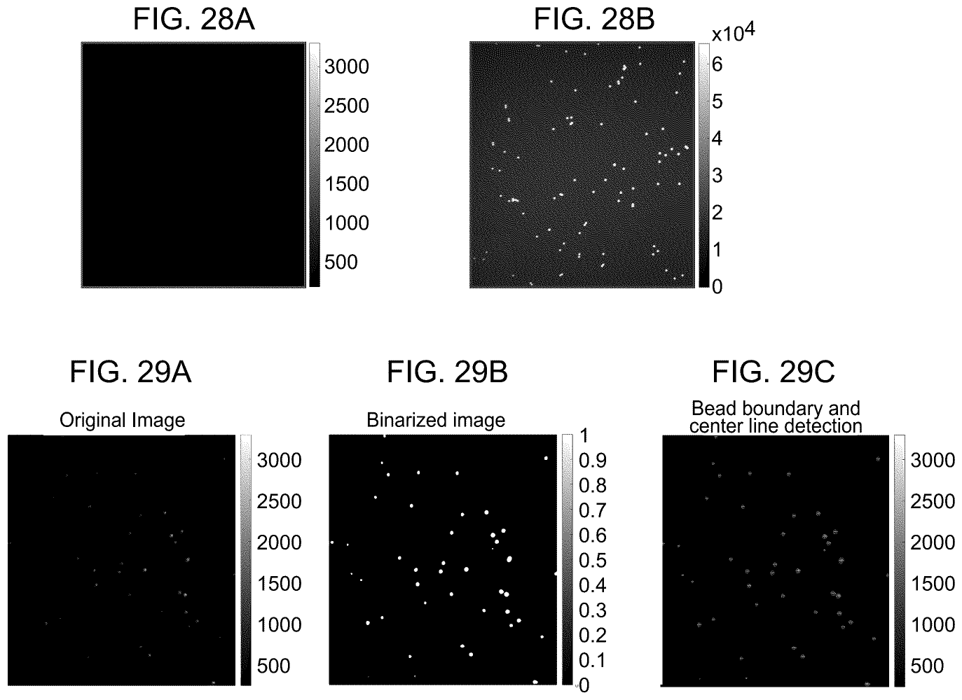

The control experiments implemented using SBDSPS (FIG. 3D-F) to negate the possibility of non-specific binding were reiterated on the benchtop as discussed in the benchtop synthesis section. The fluorescence of these beads was measured following the same procedure as discussed in the previous paragraph (FIG. 28).

Image Processing and Data Analysis

Encapsulation and Ejection of Bead from Droplet

The recorded video of the encapsulation and ejection process was analyzed frame by frame using ImageJ and snapshots that best represent the processes were selected and labelled for FIG. 2.

Establishing Enzymatic Coupling of Base to the Initiator Strands on the Bead

The image under red excitation of the oil filled device before introducing the beads is shown in FIGS. 23A and B. FIG. 23A has a grey scale of 130-250. FIG. 23B is the same as FIG. 23A but with a scale of 200-3300. FIGS. 23C and D represent the image of the bead under blue and red excitation respectively after dielectrophoretic trapping and before encapsulation and ejection from the droplet. FIGS. 23E and F represent the image of the bead under blue and red excitation respectively after encapsulation and ejection from the droplet. The three beads that represent chemical coupling are represented in FIG. 24 with a color scale of 200-3300. FIG. 25 represents the same thing as in FIG. 24 but for control experiments.

Enzymatic Coupling on Benchtop

The fluorescence images of the beads in FIGS. 27 and 28 are scale adjusted to 200-3300. The representative beads from FIG. 27 are taken by cropping areas around individual beads (FIG. 4A). Similarly, the individual beads images from SBDSPS are taken for FIG. 24 and used as FIG. 4C. Fluorescence intensities are plotted along a horizontal line passing through the center of each bead of FIGS. 4A and C in FIG. 4D.

Analyzing Fluorescence Intensity Distribution

Each frame in FIG. 27 was analyzed using predefined image processing functions in Matlab to detect the beads (FIG. 29A), binarize them, evaluate their mean fluorescence intensity (FIG. 29B), and evaluate fluorescence intensity distribution across a horizontal line passing through the bead center (FIG. 29C). Average fluorescence values across frames were collected to obtain the histogram of the fluorescence intensity distribution of all beads reacted on the benchtop synthesizer (FIG. 4B).

Analyzing Statistical Distribution of Data

To establish the statistical significance of our fluorescence comparison-based claim that the solid-phase synthesis reaction fidelity achieved using SBDSPS is higher than benchtop synthesis we resort to statistical hypothesis testing. We seek to establish that the mean fluorescence intensity of beads reacted using SBDSPS is higher than the mean fluorescence intensity of beads reacted using benchtop synthesis at significance level of 0.05 or a confidence level of 95%. The t-test which tests for the null hypothesis of equivalence of sample means for both the synthesis methods using the following test statistic⁴⁻⁴⁴ (t value) is appropriate for our purpose.

TABLE
Statistical parameters obtained from experimental data.

| Statistical Parameter | Benchtop Synthesis | SBDSPS |
|---|---|---|
| Number of samples (N) | 282 | 3 |
| Mean (μ) | 629.27 | 2057.5 |
| Standard Deviation (σ) | 373 | 209.05 |

Here, μ_{DBDR/bench} is the sample mean of the respective synthesis methods, σ_{DBDR/bench} is the sample standard deviation of the respective synthesis method, and N_{DBDR/bench} is the number of bead samples over which the mean and the standard deviation were evaluated in the respective synthesis methods. For benchtop synthesis, N_{bench}=282. This is the total number of beads that were accounted for in the histogram in FIG. 4b. For SBDSPS, N_{DBDR}=3. These are the 3 beads represented in FIG. 24. The relevant values are summarized in the table below.

$$t = \frac{{μ}_{DBDR} {μ}_{bench}}{\sqrt{\frac{{σ}_{DBDR}^{2}}{{N}_{DBDR}} + \frac{{σ}_{bench}^{2}}{{N}_{bench}}}} \qquad ( eq . 1 )$$

As the standard deviations and the number of bead samples are unequal in the two synthesis methods, we use the Welch's t-test for the statistical significance analysis^{41, 42, 45}. The degree of freedom for the Welch t-test which is given by the Welch-Satterthwaite equation is^{42, 46, 47}:

$$df = \frac{{( \frac{{σ}_{DBDR}^{2}}{{N}_{DBDR}} + \frac{{σ}_{bench}^{2}}{{N}_{bench}} )}^{2}}{\frac{{( \frac{{σ}_{DBDR}^{2}}{{N}_{DBDR}} )}^{2}}{{N}_{DBDR} - 1} + \frac{{( \frac{{σ}_{bench}^{2}}{{N}_{bench}} )}^{2}}{{N}_{bench} - 1}} \qquad ( eq . 2 )$$

The values of t and df evaluated using eq. 1 and eq. 2 are 11.6351 and 2.1381 (summarized in the table below).

TABLE
Evaluated parameters for Welch t-test.

| Hypothesis Testing Parameter | Value |
|---|---|
| t | 11.6351 |
| df | 2.1381 |
| a | 0.05 |
| t* | 4.303 |
| Power | 1 |

Therefore, 2<df=2.1381<3. Using a standard t-test table for two-tailed testing we see that if df=2 for a two-tailed significance level (a) of 0.01 the critical t-value (t*) is 9.925<t and for a two-tailed significance level (α) of 0.002 the critical t-value (t*) is 22.327>t. On the other hand, if df=3 for a two-tailed significance level (α) of 0.002 the critical t-value (t*) is 10.215<t and for a two-tailed significance level (α) of 0.001 the critical t-value (t*) is 12.924>t. Thus, we can safely say that our null hypothesis can be rejected at significant level of α=0.01 or at a confidence level of 99%. Hence, our result is significant at α=0.05 or a confidence level of 95%. This was confirmed using the inbuilt ttest2 function in Matlab for Welch's t-test which rejected the null hypothesis. To evaluate if the sample sizes (N_{DBDR} and N_{column}) were sufficient for statistical testing, we calculate the power of the statistical test⁴⁸ using the inbuilt sampsizepwr function in matlab for α=0.05. We obtain a statistical power of almost 1 (a power of 0.8 at α=0.05 is generally considered adequate⁴⁸). Therefore, our sample size suffices for statistical testing.

The Welch's t-test, which is a parametric test is generally robust for normal distributions with unequal sample sizes and standard deviations^{41, 42, 43.} For deviations from normal distributions (FIG. 4B), nonparametric tests (which do not assume any specific distribution profile) operating on the ranks of the experimentally observed values rather than the actual values themselves are more robust^{45, 49.} It is established in statistical literature that a rank transformation on the conventional Welch's t-test would counter the combined effects of unequal standard deviations as well as non-normal distributions⁴⁵. Therefore, we apply the above statistical testing procedure to the combined ranks of the average fluorescence intensities of beads reacted using the benchtop synthesis and SBDSPS. The 3 beads reacted using SBDSPS have higher fluorescence intensities than the 282 beads reacted on the benchtop platform. So, the beads reacted in benchtop have ranks from 1 to 282. While the beads reacted using SBDSPS have ranks from 283 to 285. The respective means (μ_{DBDR/column}) and standard deviations (σ_{DBDR/column}) are summarized in the table below.

TABLE
Statistical parameters obtained from rank
transformations of experimental data.

| Statistical Parameter | Benchtop Synthesis | SBDSPS |
|---|---|---|
| Number of samples (N) | 282 | 3 |
| Mean (μ) | 141.5 | 284 |
| Standard Deviation (σ) | 81.4059 | 0.8165 |

Using eq. 1 and 2, we evaluate t=29.2577 and df=282.7867. We see that at α=0.05, t*=1.9684<t=29.2577 for df=282 or 283. Therefore, the null hypothesis of equivalence of means of SBDSPS and benchtop synthesizers can be safely rejected at the confidence level of 95%. The inbuilt ttest2 function in matlab confirms this. Furthermore, a statistical power of 0.9729 is obtained which confirms that the sample size suffices for the statistical inference. The results are summarized in the table below.

TABLE
Evaluated parameters for the Welch t-test
on the rank transformed parameters.

| Hypothesis Testing Parameter | Value |
|---|---|
| t | 29.2577 |
| df | 282.7867 |
| a | 0.05 |
| t* | 1.9864 |
| power | 0.9729 |

Electrohydrodynamic Simulation of the Encapsulation and Ejection of the Bead by the Droplet

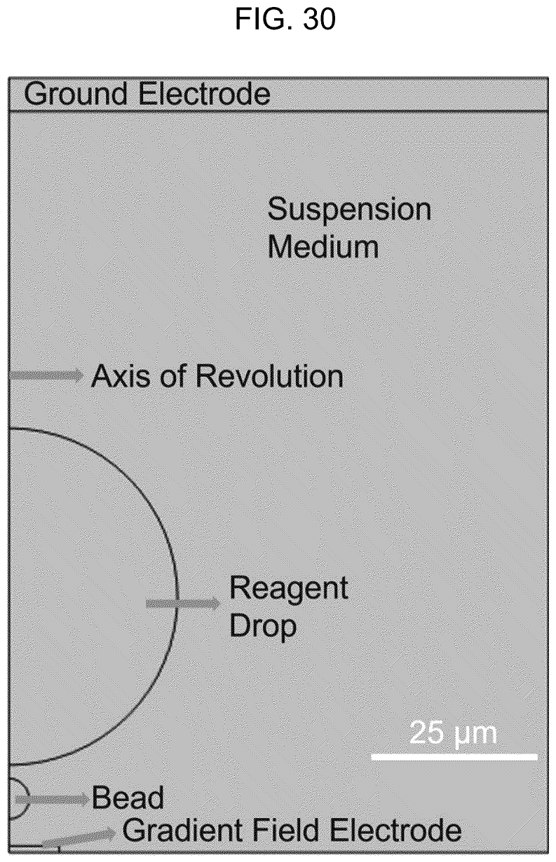

The electric field-driven engulfing of the bead by the droplet and its subsequent ejection was modeled by coupling the Navier Stokes equation for incompressible fluids (eq. 3) with the charge continuity equation of electrodynamics (eq. 4). The spatially varying electric field due to suitably defined electrodes exerts non-uniform pressure on the fluidic interface resulting in a net force on the aqueous reagent droplet suspended in a medium of silicone oil with a kinematic viscosity of 1 cSt. The electric force exerted on the fluidic interface is given by the Maxwell Stress Tensor (eq. 5). This electric force drives fluid flow as per the Navier Stokes equation (eq. 3). As a result, the fluidic interface shifts with time. The shift in fluidic interface with time is tracked by the phase-field method (eq. 6). This shifting fluidic interface is fed back into the electrical charge continuity equation (eq. 4) as a change in the material boundary and hence a change in the boundary condition. Mimicking experimental observations (FIG. 2A) the bead was kept stationary in the simulations. This also reduces the number of moving components and greatly simplifies the simulation while keeping the essential physics underlying the process intact. Furthermore, an axis-symmetric model was employed for the simulation to reduce computational resource requirements (FIG. 30) while focusing on the essential physical principles underlying the process.

$$ρ \frac{\partial \vec{u}}{\partial t} + ρ ( \vec{u} \cdot \vec{∇} ) \vec{u} = \vec{∇} \cdot [ - p \vec{I} + \overleftrightarrow{K} ] + {\vec{F}}_{electric} \qquad ( eq . 3 A )$$

$$ρ \vec{∇} \cdot \vec{u} = 0 \qquad ( eq . 3 B )$$

$$\overleftrightarrow{K} = μ \{ ( \vec{∇} \vec{u} ) + {( \vec{∇} \vec{u} )}^{T} \} \qquad ( eq . 3 C )$$

$${\vec{F}}_{electric} = \vec{∇} \cdot {{T}^{⟷}}_{electric} \qquad ( eq . 3 D )$$

Here ρ is density of the fluid, \vec{u} is velocity of fluid flow, p is pressure, [symbol] is the identity tensor, [symbol] is the viscous stress tensor, μ is the dynamic viscosity of fluid and [symbol] _{electric} is the electric component of Maxwell Stress Tensor.

$$\vec{∇} \cdot \vec{J} = - \frac{\partial {ρ}_{q}}{\partial t} \qquad ( eq . 4 A )$$

$$\overleftrightarrow{J} = σ \vec{E} \qquad ( eq . 4 B )$$

$$\vec{E} = - \vec{∇} V \qquad ( eq . 4 C )$$

$$σ = {Vf}_{r} {σ}_{r} + {Vf}_{o} {σ}_{o} and ε = {Vf}_{r} {ε}_{r} + {Vf}_{o} {ε}_{o} \qquad ( eq . 4 D )$$

Here \vec{J} is the current density, ρ_{q} is the charge density, σ is the electrical conductivity, \vec{E} is the electric field, \vec{D} is the electric displacement, εₒ is the permittivity of free space, ε is the relative permittivity, V is the electric potential, Vf_{r/o} represents the volume fraction of the reagent droplet/oil medium, σ_{r/o} and ε_{r/o} represent the electrical conductivity and the electrical permittivity of the reagent droplet/oil medium respectively.

[symbol]

=\vec{E}⊗\vec{D}−½(\vec{E}·\vec{D}) (eq. 5)

Here ⊗ represents outer product of two vectors.

$$\frac{\partial ϕ}{\partial t} + \vec{u} \cdot \vec{∇} ϕ = \vec{∇} \cdot \frac{3 {χϵ}_{pf}}{2 \sqrt{2}} \vec{∇} ψ \qquad ( eq . 6 A )$$

$$ψ = - \vec{∇} \cdot {ϵ}_{pf}^{2} \vec{∇} ϕ + ( {ϕ}^{2} - 1 ) \qquad ( eq . 6 B )$$

$${\vec{F}}_{st} = \frac{3 γ}{2 \sqrt{2} {ϵ}_{pf}} ψ \vec{∇} ϕ \qquad ( eq . 6 C )$$

$${Vf}_{o} = \frac{1 - ϕ}{2} and {Vf}_{r} = \frac{1 + ϕ}{2} \qquad ( eq . 6 D )$$

Here ϕ represents the phase field variable which is −1 in the suspension medium and 1 in the reagent droplet and transitions from −1 to 1 at the droplet-medium interface. The phase field method models the interface as a transition region of non-zero thickness over which the two fluids mix with varying volume fractions Vfᵣ and Vfₒ. ϵ_{pf} is the interfacial thickness parameter which determines the stiffness of the transition from the reagent phase (ϕ=1) to the oil phase (ϕ=−1). It should be small enough to maintain the sharpness of the interface and capture the physics accurately but large enough to prevent the finite mesh size from causing numerical instabilities. χ is called the mobility tuning parameter. It should be large enough to accurately track the shift in the interface position with fluid flow while still being small enough to give a sharp enough of an interface. γ is the surface tension coefficient.

As the droplet moves under the influence of the applied electric stress (eq. 3D and eq. 5) as per the Navier Stokes equation (eq. 3A-C) the spatial position of the droplet-medium interface evolves. The resultant evolution of the fluidic interface was tracked using the phase field method (eq. 6). The changing boundary was reflected as a change in the spatial profile of the electrical conductivity and relative permittivity (eq. 4E) which in turn modified the solution of the charge continuity equation (eq. 4A). This system of coupled equations was solved using COMSOL Multiphysics which is a commercially available finite element method-based simulation package.

Potential Energy-Based Evaluation of the Engulfing and Ejection Process

The Gibbs free energy of the fluidic interfaces is given by:

U_{IT}=γ_{ow}A_{ow}+γₒₛAₒₛ+γ_{ws}A_{ws} (eq. 7)

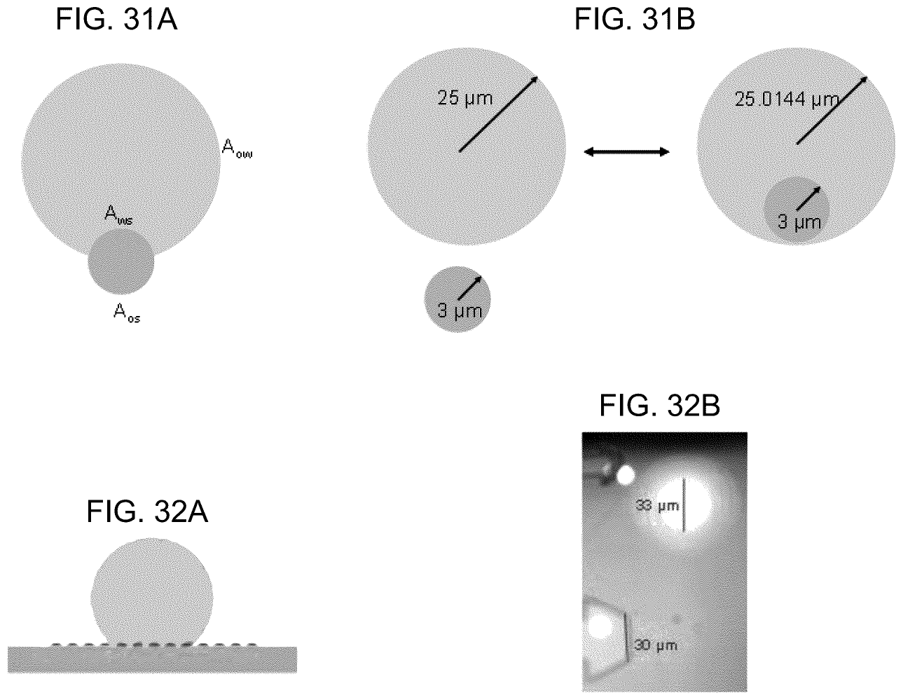

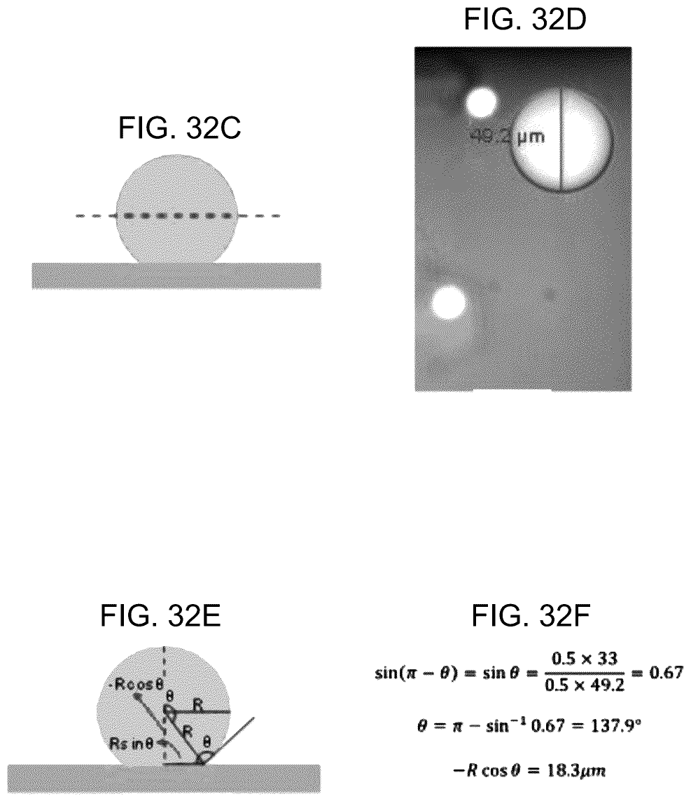

The total surface area of the bead is a constant (Aₛ) (FIG. 31). So,

Aₛ=Aₒₛ+A_{ws} (eq. 8)

The contact angle that a reagent droplet forms on the surface of the streptavidin coated bead in a medium of silicone oil is given by:

$$cos θ = \frac{{γ}_{o s} - {γ}_{w s}}{{γ}_{o w}} \qquad ( eq . 9 )$$

Using eq. 8 and eq. 9 in eq. 7 the following is obtained:

U_{IT}=γₒₛAₛ+γ_{ow}(A_{ow}−cos θA_{ws}) (eq. 10)

The first part of the right side of eq. 10 is a constant. Therefore,

ΔU_{IT}=γ_{ow}(ΔA_{ow}−cos θΔA_{ws}) (eq. 11)

Specifically for the system under consideration with r_{b}=3 μm (radius of bead) and r_{d}=25 μm (radius of droplet), as the droplet completely engulfs the bead starting from separate bead and droplet provides the following:

ΔA_{ws}=4π×3² μm² (eq. 12)

As the droplet engulfs the bead its volume remains constant and therefore its surface area with the surrounding medium increases. This can be expressed as follows:

4/3π(R_{d}+ΔR_{d})³=4/3πR_{d}³+4/3πr_{b}³ (eq. 13A)

ΔA_{ow}=4π(R_{d}+ΔR_{d})²−4πR_{d}² (eq. 13B)

From eq. 13A ΔR_{d}=0.01 μm. Therefore, ΔA_{ow}=4π×50.0144×0.0144 μm². Comparing the terms on the right side of eq. 9 the following is seen:

$$\frac{- cos θ Δ {A}_{w s}}{Δ {A}_{o w}} = 10.82 \qquad ( eq . 14 )$$

Therefore, ΔA_{ow} is neglected in the calculations. So, the following is assumed:

ΔU_{IT}≈−γ_{ow} cos θΔA_{ws} (eq. 15)

As the droplet moves the electrical energy of the system changes. That means the electrical energy stored in the circuit changes (35).

$$Δ {U}_{E} = - \frac{Δ ( Q {V}_{s} )}{2} \qquad ( eq . 16 )$$

Here Q is the charge on the electrode and Vₛ is the supply voltage on it. The total change in the energy of the system as the droplet encapsulates and ejects the bead is given by:

ΔU=ΔU_{IT}+ΔU_{E} (eq. 17)

As the droplet moves its center of mass shifts which was evaluated using the following equation for all z such that ϕ>0 in the simulation.

$${z}_{d} = \frac{Σ ρ z Δ V}{Σ ρ Δ V} \qquad ( eq . 18 )$$

The center of mass of the bead is fixed (z_{b}) in the simulations. The distance between the center of masses is then evaluated as:

Δz_{CM}=z_{d}−z_{b} (eq. 19)

The change in energies is plotted against Δz_{CM} in FIGS. 2C and D.

Padhy et al. (2022) Bead-Droplet Reactor for High-Fidelity Solid-Phase Enzymatic DNA Synthesis (arXiv:2211.06799 [physics.app-ph]) is incorporated herein by reference in its entirety for all purposes.

### REFERENCES

1. C. A. Voigt, Synthetic biology 2020-2030: six commercially-available products that are changing our world. Nat. Comm. 11, 6379 (2020).
2. A. S. Khalil, J. J. Colins, Synthetic biology: applications come of age. Nat. Rev. Genet. 11, 367-379 (2010).
3. D. R. Georgianna, S. P. Mayfield, Exploiting diversity and synthetic biology for the production of algal biofuels. Nat. 488, 329-335 (2012).
4. E. T. Wurtzel, C. E. Vickers, A. D. Hanson, A. H. Millar, M. Cooper, K. P. Voss-Fels, P. I. Nikel, T. J. Erb, Revolutionizing agriculture with synthetic biology. Nat. Plants 5, 1207-1210 (2019).
5. N. C. Seeman, Nanomaterials based on DNA. Annu. Rev. Biochem. 79, 65-87 (2010).
6. N. Goldman, P. Berton, S. Chen, C. Dessimoz, E. M. LeProust, B. Sipos, E. Birney, Towards practical, high-capacity, low-maintenance, information storage in synthesized DNA. Nat. 494, 77-80 (2013).
7. H. Lee, D. J. Weigand, K. Griswold, S. Punthambaker, H. Chun, R. E. Kohman, G. M. Church, Photon-directed multiplexed enzymatic DNA synthesis for molecular digital data storage. Nat. Comm. 11, 5246 (2020).
8. S. Kosuri, G. M. Church, Large-scale de novo DNA synthesis: technologies and applications. Nat. Methods 11, 499-507 (2014).
9. R. A. Hughes, A. D. Ellington, Synthetic DNA synthesis and assembly: putting the synthetic in synthetic biology. Cold Spring Harb. Perspect. Biol. 9, a023812 (2017).
10. S. Ma, N. Tang, J. Tian, DNA synthesis, assembly and applications in synthetic biology. Curr. Opin. Chem. Biol. 16, 260-267 (2012).
11. M. Eisenstein, Enzymatic DNA synthesis enters new phase. Nat. Biotechnol. 38, 1113-1115 (2020).
12. N. Ostrov, J. Beal, T. Ellis, D. B. Gordon, B. J. Karas, H. H. Lee, S. C. Lenaghan, J. A. Schloss, G. Stracquadanio, A. Trefzer, J. S. Bader, G. M. Church, C. M. Coelho, J. W. Efcavitch, M. Güell, L. A. Mitchell, A. A. K. Nielsen, B. Peck, A. C. Smith, C. N. Stewart Jr., H. Tekotte, Technological challenges and milestones for writing genomes, Science 366, 310-312 (2019).
13. S. Ma, I. Saaem, J. Tian, Error correction in gene synthesis technology, Trends Biotechnol. 30, 147-154 (2012).
14. M. A. Jensen, R. W. Davis, Template independent enzymatic oligonucleotide synthesis (TiEOS): Its history, prospects and challenges. Biochemistry 57, 1821-1832 (2018).
15. R. B. Merrifield, Solid Phase Synthesis. Science 232, 341-357 (1986).
16. K. S. Elvira, X. C. I. Solvas, R. C. R. Wootton, A. J. DeMello, The past, present and potential of microfluidic reactor technology in chemical synthesis. Nat. Chem. 5, 905-915 (2013).
17. I. C. Clark, A. R. Abate, Microfluidic bead encapsulation above 20 kHz with triggered drop formation. Lab Chip 18, 3598-3605 (2018).
18. A. R. Abate, C. Chen, J. J. Agresti, D. A. Weitz, Beating Poisson encapsulation statistics using close-packed ordering. Lab Chip 9, 2628-2631 (2009).
19. P. C. Gach, K. Iwai, P. W. Kim, N. J. Hillson, A. K. Singh, Droplet microfluidics for synthetic biology. Lab Chip 17, 3388-3400 (2017).
20. R. Pethig, Dielectrophoresis: status of the theory, technology and applications. Biomicrofluidics 4, 022811 (2010).
21. F. F. Becker, X. B. Wang, Y. Huang, R. Pethig, J. Vykoukal, P. R. Gascoyne, Separation of human breast cancer cells from blood by differential dielectric affinity. Proc. Natl. Acad. Sci. 92, 860-864 (1995).
22. O. D. Velev, B. G. Prevo, K. H. Bhatt, On-chip manipulation of free droplets, Nat. 426, 515-516 (2003).
23. J. R. Millman, K. H. Bhatt, B. G. Prevo, O. D. Velev, Anisotropic particle synthesis in dielectrophoretically controlled microdroplet reactors. Nat. Mater. 4, 98-102 (2005).
24. K. D. Hermanson, S. O. Lumsdon, J. P. Williams, E. W. Kaler, O. D. Velev, Dielectrophoreic assembly of electrically functional microwires from nanoparticle suspensions. Science 294, 1082-1086 (2001).
25. P. Zhu, L. Wang, Passive and active droplet generation with microfluidics: a review. Lab Chip 17, 34-75 (2017).
26. F. Mugele, J. C. Baret, Electrowetting: from basics to applications. J. Phys. Condens. Matter. 17, R705-R774 (2005).
27. D. T. Chiu, R. M. Lorenz, G. D. M. Jeffries, Droplets for ultrasmall-volume analysis, Anal. Chem. 81, 5111-5118 (2009).
28. A. M. Nightingale, T. W. Phillips, J. H. Banock, J. C. DeMello, Controlled multistep synthesis in a three-phase droplet reactor. Nat. Comm. 5, 3777 (2014).
29. J. H. Banock, S. H. Krishnadasan, A. M. Nightingale, C. P. Yau, K. Khaw, D. Burkitt, J. J. M. Halls, M. Heeney, J. C. DeMello, Continuous synthesis of device-grade semiconducting polymers in droplet based microreactors. Adv. Func. Mater. 23, 2123-2129 (2013).
30. P. Y. Chiou, A. T. Ohta, M. C. Wu, Massively parallel manipulation of single cells and microparticles using optical images. Nat. 436, 370-372 (2005).
31. A. Kuchler, M. Yoshimoto, S. Luginbuhl, F. Mavelli, P. Walde, Enzymatic reactions in confined environments. Nat. Nanotechnol. 11, 409-420 (2016).
32. M. Prakash, N. Gershenfeld, Microfluidic Bubble Logic. Science 315, 832-835 (2007).
33. K. S. Lam, M. Lebl, V. Krchňák, The one-bead-one-compound combinatorial library method. Chem. Rev. 97, 411-448 (1997).
34. P. Garstecki, M. J. Fuertsman, H. A. Stone, G. M. Whitesides, Formation of droplets and bubbles in a microfluidic T-junction-scaling and mechanism of break up. Lab Chip 6, 437-446 (2006).
35. P. Padhy, M. A. Zaman, M. A. Jensen, L. Hesselink, Dynamically controlled dielectrophoresis using resonant tuning. Electrophoresis 42, 1079-1092 (2021).
36. S. Michal, N. Hana, K. Jan, The determination of viscosity at liquid mixtures-comparison of approaches. AIP Conf. Proc. 1889, 020035 (2017).
37. L. Cui, D. Holmes, H. Morgan, The dielectrophoretic levitation and separation of latex beads in microchips. Electrophoresis 22, 3893-3901 (2001).
38. S. Torza, R. G. Cox, S. G. Mason, Electrohydrodynamic deformation and bursts of liquid drops. Philos. Trans. Royal Soc. A 269, 295-319 (1971).
39. A. Mikkelsen, A. Kertmen, K. Khobaib, M. Rajnak, J. Kurimsky, Z. Rozynek, Assembly of 1D granular structures from sulfonated polystyrene particles, Materials 10, 1212 (2017).
40. Krzywinski, M., & Altman, N., Significance, p values and t-tests. Nat. Methods 10, 1041-1042 (2013).
41. Krzywinski, M., & Altman, N., Comparing samples—part 1. Nat. Methods 11, 215-216 (2014).
42. Derrick, B., Toher, D., & White, P., Why Welch's t-test is type I error robust. Quant. Meth. Psych. 12, 30-38 (2016).
43. Ross, A., Willson, & V. L., Basic and advanced statistical tests: Writing results sections and creating tables and figures. Sense Publishers, Rotterdam (2017).
44. Schumacker, R., & Tomek, S., Understanding statistics using R. Springer, New York (2013).
45. Zimmerman, D. W., & Zumbo, B., D., Rank transformations and the power of the student t test and welch t′ test for non-normal populations with unequal variances. Can. J. Exp. Psychol. 47, 523-539 (1993).
46. Miles, J., & Banyard, P., Understanding and using statistics in psychology: a practical introduction. Sage Publications, London (2007).
47. Alfassi, Z. B., Boger, Z., & Ronen, Y., Statistical treatment of analytical data. CRC Press, Boca Raton (2005).
48. Krzywinski, M., & Altman, N., Power and sample size. Nat. Methods 10, 1139-1140 (2013).
49. Krzywinski, M., & Altman, N., Nonparametric tests. Nat. Methods 11, 467-468 (2013).

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

1.-38. (canceled)

39. A method of engulfing a particle into a droplet, the method using a microfluidic device comprising a droplet generator and a chamber, the chamber comprising a liquid medium disposed on a voltage supply electrode and a ground electrode, wherein the method comprises:
(a) dispensing a particle and a droplet into the liquid medium;
(b) dielectrophoretically trapping the particle and the droplet using the voltage supply electrode and the ground electrode; and
(c) engulfing the particle into the droplet, wherein the engulfing comprises increasing a supply voltage between the voltage supply electrode and the ground electrode, thereby moving the droplet toward the voltage supply electrode and engulfing the particle into the droplet.

40. The method according to claim 39, wherein the liquid medium comprises, consists essentially of, or consists of an oil.

41. The method according to claim 39, wherein voltage supply electrode is transparent.

42. The method according to claim 39, wherein voltage supply electrode is an indium tin oxide (ITO) electrode.

43. The method according to claim 39, wherein the particle is a microparticle or a cell.

44. The method according to claim 39, wherein the method further comprises modifying the engulfed particle.

45. The method according to claim 44, wherein the modifying comprises covalently or non-covalently attaching a molecule present in the droplet to the particle.

46. The method according to claim 45, wherein the modifying comprises covalently or non-covalently attaching a nucleotide or polynucleotide present in the droplet to the particle.

47. The method according to claim 46, wherein the attaching comprises base-pairing the nucleotide or polynucleotide to a nucleotide or polynucleotide present on the particle prior to step (d).

48. The method according to claim 45, wherein the modifying comprises covalently or non-covalently attaching an amino acid, polypeptide, sugar or carbohydrate present in the droplet to the particle.

49. The method according to claim 39, wherein the method further comprises assessing the engulfed particle.

50. The method according to claim 49, wherein the particle is a cell, and wherein the method comprises assessing the cell for expression of a polypeptide.

51. The method according to claim 39, wherein the method further comprises:
(d) decreasing the supply voltage to eject the particle from the droplet.

52. The method according to claim 51, further comprising subjecting the ejected particle to steps (b) and (c) to re-engulf the particle in a droplet.

53. The method according to claim 39, comprising imaging the particle during one or more or more of steps (a)-(c).

54. A microfluidic device comprising:
a chamber adapted to contain a liquid medium;
a fluidic port operably coupled to the chamber;
a droplet generator operably coupled to the chamber;
a voltage supply electrode and a ground electrode disposed on a surface of the chamber, wherein the voltage supply electrode and a ground electrode are aligned with the droplet generator for controlled manipulation of a droplet and a particle when the device is in use.

55. The microfluidic device of claim 54, wherein the device is operably coupled to a fluidic subsystem, wherein the fluidic subsystem comprises a fluidic tank comprising an output fluidically coupled to the chamber when the device is in use.

56. The microfluidic device of claim 55, wherein the fluidic subsystem further comprises a pressure controller operably coupled to a fluidic tank.

57. The microfluidic device of claim 54, wherein the voltage supply electrode and the ground electrode are operably coupled to an electrical subsystem comprising a function generator and an amplifier.

58. The microfluidic device of claim 54, wherein the device is operably coupled to an optical subsystem comprising a microscope.
